(19) United States
(12) Patent Application Publication
(10) Pub. No.: US 2024/0345236 A1
(43) Pub. Date: Oct. 17, 2024

(54) SCANNING SYSTEM AND METHOD

(71) Applicant: GLOUCHESTER HOSPITALS NHS FOUNDATION TRUST

(72) Inventor: Alexander GILES, London (GB)

(73) Assignee: GLOUCHESTER HOSPITALS NHS FOUNDATION TRUST, Gloucester (GB)

(21) Appl. No.: 18/627,587

(22) Filed: Apr. 5, 2024

(30) Foreign Application Priority Data
Apr. 6, 2023 (GB) 2305146.9

(51) Int. Cl. G01S 13/02; A61B 5/05; G01S 7/282; G01S 13/10; G01S 13/522

(52) U.S. Cl. CPC G01S 13/0209; A61B 5/05; G01S 7/282; G01S 13/106; G01S 13/522

(57) ABSTRACT

A system for scanning a subject is provided, the system comprising: a transmitter operable to emit a train of pulses comprising a plurality of ultra-wide band (UWB) radio-wave pulses; and a receiver operable to receive reflected radio-wave pulses corresponding to each pulse of the plurality of pulses emitted by the transmitter, the receiver operable to: detect a reflected pulse for the first pulse of the plurality of pulses a time period T₁ seconds after the first pulse P₁ has been emitted; and thereafter to detect a reflected pulse for each subsequent emitted pulse Pₘ, where m is the number of the pulse, a time period Tₘ seconds after the pulse Pₘ has been emitted, where Tₘ is longer than the time period Tₘ₋₁for the preceding pulse Pₘ₋₁; and a control unit comprising a processor operable to receive data from the receiver relating to the reflected pulses detected. A method of scanning a subject is also provided, the method comprising: transmitting a train of pulses comprising a plurality of ultra-wide band (UWB) radio-wave pulses at the subject or an expected location of the subject; detecting a reflected pulse for the first pulse of the plurality of pulses a time period T₁ seconds after the first pulse P₁ has been emitted; detecting a reflected pulse for each subsequent emitted pulse Pₘ, where m is the number of the pulse, a time period Tₘ seconds after the pulse Pₘ has been emitted, wherein Tₘ is longer than the time period Tₘ₋₁ for the preceding pulse Pₘ₋₁; and analysing the reflected pulses detected. The system and method find particular use in the detection of movement of a subject, more especially in monitoring the respiration rate or heart rate of the subject.

The present invention relates to a system for scanning a subject. The system finds use in locating a subject. The system also finds use in monitoring physiological signs of a subject. The present invention also relates to a method for scanning a subject. Again, the method finds use in locating a subject and for detecting and monitoring physiological signs of a subject, for example a subject undergoing medical care and treatment.

Systems and techniques for locating subjects are known in the art. For example, real-time locating systems (RTLS), also known as real-time tracking systems, are known in the art and are used to identify and track the location of objects or subjects in real time. RTLSs are typically employed within a building or other confined area. In operation, wireless RTLS tags are attached to the objects or subjects to be monitored. Fixed RTLS stations receive wireless signals from the tags to determine their location and the objects or subject to which they are attached. Examples of the use of RTLSs include tracking vehicles as they progress through an assembly line, locating items of merchandise in a storage facility, such as a warehouse, or monitoring the location of medical equipment or subjects, including medical personnel and patients, in a hospital. RTLSs typically employ radio frequency signals to communicate between the monitoring station and the objects or subjects being monitored.

ISO/IEC 24730-1:2014; Information Technology—Real-Time Locating Systems (RTLS)—Part 1: Application Program Interface, (API). ISO: Geneva, Switzerland, 2006, defines Real Time Locating Systems as being wireless systems with the ability to locate the position of an item anywhere in a defined space (local/campus, wide area/regional, global) at a point in time that is, or is close to, real time. Position is derived by measurements of the physical properties of the radio link.

Mahfouz, M., et al., ‘Recent Trends and Advances in UWB Positioning’, IEEE MTT-S International Microwave Workshop on Wireless Sensing, Local Positioning, and RFID (IMWS 2009—Croatia), report on developments in the use of ultra-wide band (UWB) technology for use in asset tracking and navigation, including surgical navigation. It is concluded that a need exists for high accuracy UWB positioning systems (for example having 1 mm 3-D accuracy) for a new set of applications including surgical navigation.

Kamel Boulos, M. N., et al., ‘Real-time locating systems (RTLS) in healthcare: a condensed primer’, International Journal of Health Geographics, 2012, 11:25, report that a number of RTLS technologies have been used to solve indoor tracking problems. The ability to accurately track the location of assets and individuals indoors has many applications in healthcare. A summary of the use of RTLS in healthcare applications is provided, briefly covering the many options and technologies that are involved, as well as the various possible applications of RTLS in healthcare facilities and their potential benefits, including capital expenditure reduction and workflow and patient throughput improvements.

Supatra, M., et al., ‘On performance study of UWB real time locating system’, 7th International Conference of Information and Communication Technology for Embedded Systems (IC-ICTES), 2016, discuss the results of a performance study on the location accuracy of DecaWave's TREK1000, a two-way ranging RTLS IC evaluation kit. based on the use of ultra-wide band (UWB) ranging technology. It is reported that the kit tested showed a promising potential for micro-location applications. However, some limitations in the performance of UWB RTLS in both 2D and 3D cases was found. On average, the 2D accuracy was found to be within the sub-meter level. However, the 3D accuracy could be worse, up to 3-meter level. A number of guidelines for deploying the UWB RTLS properly to achieve a good localization performance are provided, such as where the anchors should be installed and the suitable area the tag should be within. It was concluded that the need to improve location accuracy along the z-axis was an important issue to be investigated further in the future.

Šinko, S., et al., ‘Performance-Oriented UWB RTLS Decision-Making Approach’, Sustainability, 2022, 14, 11456, provides a review of factors to consider when selecting a real-time location system (RTLS) based on Ultra-wideband (UWB) technology in the indoor work environment.

WO 2015/063488 discloses a method and system for the passive sensing of a subject. The method uses wireless digital communications, such as WiFi, WiMax or LTE. The wireless digital communications are frame-based, with a predefined frame structure. The method includes receiving a reference signal into a reference channel, wherein the reference signal comprises a direct version of a radio frequency transmission as part of the wireless digital communications. A surveillance signal is received into a surveillance channel. Portions of the reference signal corresponding to data transmissions based on the predefined frame structure are detected and extracted. Portions of the surveillance signal corresponding to the extracted portions of the reference signal are extracted. A cross-correlation on the extracted portions of the reference signal and the surveillance signal is performed to determine a range-Doppler surface. A real-time display of said range-Doppler surface and/or of information derived therefrom is then provided. This method exploits the known, predefined frame structure to locate those portions of the wireless digital communications that are most effective for performing passive sensing. In some embodiments, detecting and extracting portions of the reference signal includes detecting locations of a predefined synchronisation sequence in the reference signal. In many cases, identification of the predefined synchronisation sequence also allows a receiver to determine the particular communications format and its associated frame structure. The receiver can then exploit this knowledge to decode the actual content of the wireless digital communications, for example, to access information such as frame size, and the like. In some embodiments, detecting and extracting portions of the reference signal includes the receiver using the detected locations of the predefined synchronisation sequence in the reference signal to determine portions of the reference signal to extract.

There is a need for an improved system and technique for locating subjects in a range of environments and situations. It would be particularly advantageous if the system could operate without first providing the subject with a tag or other device with which to communicate, that is be a passive system. It would also be advantageous if the system could be simple to construct and operate, using a minimum of components, and able to be configured into a compact device, that is readily transported, most preferably being handheld. Such a system would have considerable applications, for example in locating subjects in accident and emergency situations, disaster locations, and generally any situation where there is a need to locate one or more persons. Such a system would also find considerable applications in the healthcare sector, in particular with the monitoring of patients.

Techniques for detecting the movement of a subject that do not rely on the subject being provided with a tag or other device are known in the art.

Braga, A. J., et al., ‘An Ultra-Wideband Radar System for Through-the-Wall Imaging Using a Mobile Robot’, 2009 IEEE International Conference on Communications, Dresden, Germany, 2009, pages 1 to 6, discuss the use of an UWB radar system for imaging objects through walls and other visually opaque structures or materials.

Singh, S., et al., ‘Sense through wall human detection using UWB Radar’, EURASIP Journal on Wireless Communications and Networking, 2011, 2011:20, discuss a system for detection of a stationary human target behind a wall based on breathing movements of the subject. In detecting the breathing motion, a Doppler based method is used. Analysis of the received signals using short time Fourier transform is suggested. However, it is reported that this did not reliably provide an indication of the respiration rate of the subject.

Saad, M., et al., ‘Development of an IR-UWB Radar System for High-Resolution Through-Wall Imaging’, Progress In Electromagnetics Research C, Vol. 124, 2022, pages 81 to 96, disclose a radar system for through-wall imaging using an impulse-radio ultra-wideband (IR-UWB) signal. The radar system transmits impulse signals having a monocycle shape with a 400-picosecond duration and a 4.6 GHz bandwidth.

Lauteslager, T., et al., ‘Validation of a New Contactless and Continuous Respiratory Rate Monitoring Device Based on Ultra-Wideband Radar Technology’, Sensors, 2021, 21, 4027, describe and assess the the Circadia Contactless Breathing Monitor™ (model C100). This device uses ultra-wideband radar to monitor the respiratory rate of a subject.

Tiberi, G., et al., ‘Ultra-Wideband (UWB) Systems in Biomedical Sensing’, Sensors 2022, 22, provide a brief review of UWB systems for the non-invasive monitoring of subjects movements, such as breathing and coughing, as well as determining the posture of the subject.

U.S. Pat. No. 5,757,320 discloses a short range, ultra-wideband radar system with a high resolution swept range gate. The system is used for range finding and the detection of hidden objects.

Animal health and wellness monitoring using ultra-wideband (UWB) radar is disclosed in US 2017/0181409.

A pulsed radar system is described and shown in US 2020/0003881.

US 2022/0206111 discloses a non-contact method for detecting and distinguishing between humans and animals based on an impulse radio ultra-wideband (IR-UWB) bio-radar signal.

JP 2012108109 discloses certain ultra-wideband (UWB) pulse sensors.

Despite the ongoing development of systems and techniques for locating subjects and monitoring their movement patterns, there is a need for an improved system and method. It would be advantageous if the improved system and method could provide a high level of penetration through structures, while at the same time providing the sensitivity required to identify small movement patterns of the subject. It would also be advantageous if the system could be arranged in a compact manner, in particular to be readily portable.

According to a first aspect of the present invention there is provided a system for scanning a subject comprising:

a transmitter operable to emit a train of pulses comprising a plurality of ultra-wide band (UWB) radio-wave pulses; and

a receiver operable to receive reflected radio-wave pulses corresponding to each pulse of the plurality of pulses emitted by the transmitter, the receiver operable to:

detect a reflected pulse for the first pulse of the plurality of pulses a time period T₁ seconds after the first pulse P₁ has been emitted; and

thereafter to detect a reflected pulse for each subsequent emitted pulse Pₘ, where m is the number of the pulse, a time period Tₘ seconds after the pulse Pₘ has been emitted, where Tₘ is longer than the time period Tₘ₋₁ for the preceding pulse Pₘ₋₁;

and

a control unit comprising a processor operable to receive data from the receiver relating to the reflected pulses detected.

In a further aspect, the present invention provides a method of scanning a subject, the method comprising:

emitting a plurality of ultra-wide band (UWB) radio-wave pulses at the subject or an expected location of the subject;

detecting a reflected pulse for the first pulse of the plurality of pulses a time period T₁ seconds after the first pulse P₁ has been emitted;

detecting a reflected pulse for each subsequent emitted pulse Pₘ, where m is the number of the pulse, a time period Tₘ seconds after the pulse Pₘ has been emitted, wherein Tₘ is longer than the time period Tₘ₋₁ for the preceding pulse Pₘ₋₁; and

analysing the reflected pulses detected.

It has been found that the present invention allows a subject to be scanned effectively and quickly. In particular, the present invention allows a subject to be scanned without needing to provide the subject with a device, such as an RFID tag of the like, as required for the known RTLS technology.

In particular, the present invention allows a subject to be located in conditions or situations where the visibility of the subject is impaired or prevented. For example, the subject may be within a structure, such as a building or part thereof, for example a room, in which case the present invention allows scanning of the subject from outside the structure. Similarly, the present invention allows a subject to be scanned and located in other situations where visibility is impaired, for example in cases of a location or building being obscured by smoke or dust.

The present invention functions by detecting movement of the subject. Therefore, a person can be detected at a location due to their movements, for example walking or running. However, the ability of the present invention to detect movement of the subject also provides significant advantages in monitoring a subject, in particular monitoring their physiological signs. More particularly, the present invention allows for a subject to be scanned and their physiological signs related to movement of the subject's body, such as heartbeat or respiration, monitored. This aspect is particularly useful in the healthcare sector, where the health of a subject is required to be monitored, for example in a hospital, clinic or other medical environment. In this respect, it is to be noted that the present invention allows the subject to be scanned and their physiological signs monitored in an entirely non-invasive manner without any contact with the subject being required.

It is also a particular advantage of the present invention that the components of the system may be arranged in a compact manner, in particular in the form of a hand-held scanner, which is easy to store, transport, deploy and use in a wide range of situations.

The present invention finds use in the detection and monitoring of subjects. In this respect, while the majority of applications of the invention are in relation to human subjects, the same principles of detection and monitoring also apply to animal subjects. References herein to ‘subject’ or ‘subjects’ are to be understood as including both human and animal subjects.

The present invention employs a transmitter to emit a train of pulses comprising a plurality of ultra-wide band (UWB) radio wave pulses. In use, the plurality of pulses is targeted at the subject, for example to detect and monitor physiological signs of the subject, including heartbeat and/or respiration. Alternatively, in cases where the location of a subject is being sought, the plurality of radio wave pulses is emitted towards the region or location where the subject is present, likely to be present or possibly could be present. In use, the plurality of pulses is emitted by the transmitter. Reflected pulses are detected by the receiver, and handled in a timed, ‘strobed’ or ‘pulsed’ manner, as described in more detail below. By repeating this cycle of pulse emission and pulsed detection over a period of time, the presence and movement of the subject being targeted can be detected by analysis of the detected reflected pulses. The field of view of the system is determined by the emission energy of the transmitter antenna and the sensitivity of the receiver antenna.

As noted above, the system and method operate by emitting a train comprising plurality of radio wave pulses towards the subject or their expected location. Each pulse of the train of radio wave pulses may have any suitable duration. In particular, the duration of each pulse may be any suitable duration that provides the signal data necessary to obtain the desired indications regarding the movement of the subject, for example their location or their physiological signs, such as heart rate and respiration rate.

The duration of each pulse may be selected according to a number of factors, for example the distance of the subject from the system, the size of the subject, the nature of the motion of the subject. The pulse duration may also be varied according to the physiological signs to be detected. As a result, it is preferable the system allows the length of the pulse to be varied, for example manually by the user.

The optimal pulse duration will be determined by factors including the range resolution to be obtained and the signal-to-noise ratio (SNR). In general, the range resolution of the system is related to the duration of the pulse, with shorter pulses providing better range resolution. Longer pulses have higher energy than shorter pulses and are subject to higher SNR. The optimal pulse duration for a particular situation may be determined as follows:

$$PD = TBP / B$$

where PD is the pulse duration (seconds), TBP is the time-bandwidth product and B is the frequency bandwidth of the system.

The TBP may be calculated based on the desired range resolution and the allowable SNR.

Each pulse may have a duration of less than 100 picoseconds, preferably less than 10 picoseconds, more preferably less than 5 picoseconds, still more preferably less than 4 picoseconds, more preferably still less than 3 picoseconds, especially 2 or less picoseconds. In a preferred embodiment, each pulse has a duration of about 1 picosecond. Preferably, the pulses in the train of pulses have substantially the same duration.

Each pulse is an ultra-wide band (UWB) pulse. The frequency of the radio wave pulses used in the present invention has been selected to provide sufficient penetration through artifacts and objects, while also providing sufficient sensitivity for detecting and recognising movement patterns of the subject. In general, lower frequency pulses exhibit greater penetration through the air, objects, such as walls, and subjects. However, the sensitivity of reflected signals at lower frequencies is poor. Conversely, higher sensitivities are achievable using higher frequency pulses, but with significantly reduced penetration. Higher frequencies also introduce increased amounts of noise in the reflected signal, in turn placing a greater burden on the signal processing. It has been found that an effective penetration distance with sufficient sensitivity for allowing subject location and detection and monitoring of physiological signs of the subject, with sufficiently low levels of noise in the reflected signal, can be achieved using pulses having a frequency of from 5 to 9 GHz, more preferably from 5.5 to 8.5 GHz, still more preferably a frequency in the range of from 6 to 8 GHz, especially in the range of from 7 to 7.5 GHz. A frequency of about 7.3 GHz is preferable for many embodiments of the present invention.

The UWB radio pulses having the aforementioned duration and frequency have a low power. This ensures the system and method of the present invention are suitable and safe for use in detecting and monitoring human subjects. Typically, the power of each pulse will be of the order of 1 picoWatt or less.

In the present invention, UWB radio waves are emitted in a train comprising a plurality of UWB radio wave pulses. The pulses may be emitted at any suitable rate. Preferably, the pulses are emitted at a rate of up to one pulse every 100 nanoseconds, more preferably up to one pulse every 80 nanoseconds, still more preferably up to one pulse every 60 nanoseconds, more preferably still up to one pulse every 50 nanoseconds, especially up to one pulse every 40 nanoseconds, more especially up to one pulse every 30 nanoseconds, still more especially up to one pulse every 25 nanoseconds. Preferably, the pulses are emitted at a rate of from one pulse every 2 nanoseconds, more preferably from one pulse every 5 nanoseconds, still more preferably from one pulse every 6 nanoseconds, more preferably still from one pulse every 8 nanoseconds, especially from one pulse every 10 nanoseconds, more especially from one pulse every 12 nanoseconds, still more especially from one pulse every 15 nanoseconds. In one preferred embodiment, the pulses are emitted at a rate of from one pulse every 2 to 100 nanoseconds, more preferably from one pulse every 5 to 80 nanoseconds, still more preferably from one pulse every 6 to 60 nanoseconds, more preferably still from one pulse every 8 to 50 nanoseconds, especially from one pulse every 10 to 40 nanoseconds, more especially from one pulse every 12 to 30 nanoseconds, still more especially from one pulse every 15 to 25 nanoseconds. A pulse rate of about one pulse every 20 nanoseconds is preferred for many embodiments. In addition to emitting a train of UWB radio wave pulses, the system operates to receive and detect reflected pulses returning to the system. Pulses are reflected from the surfaces of objects, including the subject being located or monitored. Analysis of the detected reflected pulses reveals the presence of the subject and also allows for movement of the subject and physiological signs of the subject, such as heart rate and respiration rate, to be determined.

The detection of reflected pulses is carried out intermittently over a period of time, in particular using a phase-controlled or pulsed detection regime, the result of which is to provide a plurality of sequentially timed samples of the reflected pulses. In particular, the receiver is operated at a specific time after each pulse has been emitted to detect any reflected pulse. The delay before actively detecting a reflected pulse after the emitted pulse is increased incrementally for each successive pulse. A pulse is reflected if it encounters a surface. In this way, data relating to objects lying in the region extending away from the point of emission of the pulses can be gathered and processed.

In particular, the system operates to detect a reflected pulse for the first pulse P₁ of the plurality of pulses a time period T₁ seconds after the first pulse P₁ has been emitted. In this way, after T₁ seconds, the system is operable to detect whether a pulse has been reflected from the surface of an object or a subject. Such a surface will be at a location half the distance travelled by the emitted radio wave pulse in the time T₁ seconds. In the case of the emitted pulse travelling through air, the distance of the said surface will be approximately (T₁/2)×(3×10⁸) metres from the point of emission of the pulse from the system.

The length of the detection period is determined by the time interval between consecutive pulses, referred to as the pulse repetition interval (PRI). The PRI is determined by the maximum range of the system and the desired range resolution. In general, once the detection of reflected pulses has started at a time T seconds after the pulse is emitted, the duration of the detection period will be equal to the PRI minus the time delay T between the pulse being transmitted and the start of the detection period.

The detection period DP may be calculated using the following equation:

$$DP = PRI - ( 2 \times D / c ) - T$$

where DP is the detection period in seconds, PRI is the pulse repetition interval in seconds, D is the distance of the subject from the system (m); c is the speed of light (m/s) and T is the delay in seconds from the pulse being emitted to the start of the detection period.

The length of the detection period may be varied, depending upon factors such as the nature of the movement of the subject, Doppler shifts and noise in the signal.

In general, the duration of the detection period may be from 10 nanoseconds, preferably from 20 nanoseconds, more preferably from 30 nanoseconds, still more preferably from 40 nanoseconds, more preferably still from 50 nanoseconds, especially from 60 nanoseconds. The duration of the detection period may be from 70 nanoseconds, preferably from 80 nanoseconds are applicable for many embodiments. The duration may be up to 900 nanoseconds, preferably up to 800 nanoseconds, more preferably up to 700 nanoseconds, still more preferably up to 600 nanoseconds, more preferably still up to 500 nanoseconds, especially up to 400 nanoseconds. Durations from 30 to 700 nanoseconds, preferably from 50 to 500 nanoseconds are applicable for many embodiments.

As noted above, detection of the reflected pulses is started after a period T₁ seconds has elapsed from the transmission of the pulse. The time period T₁ seconds may be any suitable period, the selection of which will determine the range of field of view of the system. T₁ is preferably up to 100 nanoseconds, more preferably up to 80 nanoseconds, still more preferably up to 60 nanoseconds, more preferably still up to 50 nanoseconds, especially up to 40 nanoseconds, more especially up to 30 nanoseconds, more especially still up to 20 nanoseconds, still more especially up to 10 nanoseconds. A time for T₁ of up to 10 nanoseconds is preferred for many embodiments, more preferably up to 8 nanoseconds, still more preferably up to 6 nanoseconds, more preferably still up to 5 nanoseconds, especially up to 4 nanoseconds, more especially up to 3 nanoseconds. T₁ is preferably from 0.1 nanoseconds, more preferably from 0.3 nanoseconds, still more preferably from 0.5 nanoseconds, more preferably still from 0.75 nanoseconds, especially from 1 nanosecond, more especially from 1.2 nanoseconds, more especially still from 1.4 nanoseconds, still more especially from 1.5 nanoseconds. A time for T₁ of from 1.5 nanoseconds is preferred for many embodiments, more preferably from 1.6 nanoseconds, still more preferably from 1.7 nanoseconds, more preferably still from 1.8 nanoseconds, especially from 1.9 nanoseconds. A time T₁ of from 1.5 to 2.5 nanoseconds is preferred for many embodiments, more preferably from 1.6 to 2.4 nanoseconds, still more preferably from 1.7 to 2.3 nanoseconds, more preferably still from 1.8 to 2.2 nanoseconds, especially from 1.9 to 2.1 nanoseconds. A time T₁ of about 2 nanoseconds is preferred for many embodiments.

Thereafter, the system is operable to detect a reflected pulse for each subsequent emitted pulse Pₘ, where ‘m’ is the number of the pulse, a time period Tₘ seconds after the pulse Pₘ has been emitted, wherein Tₘ is longer than the time period Tₘ₋₁ for the preceding pulse Pₘ₋₁.

Therefore, for the second emitted pulse P₂, the system operates to detect a reflected pulse at a time T₂ seconds after the pulse P₂ has been emitted, wherein the time period T₂ is longer than the time period T₁. In this way, after T₂ seconds, the system is operable to detect a reflection of the pulse P₂ that has been reflected from the surface of an object or a subject. Such a surface will be at a location half the distance travelled by the emitted radio wave pulse in the time T₂ seconds. In the case of the emitted pulse travelling through air, the distance of the said surface will be approximately (T₂/2)×(3×10⁸) metres from the point of emission of the pulse P₂ from the system, that is further away from the point of emission than the reflected pulse from the first pulse P₁.

The time period T₂ may have the values indicated above for the time period T₁, with the proviso that T₂ is longer than T₁. The time increment ΔT₁₋₂ between T₁ and T₂ may be any suitable period. Preferably, ΔT₁₋₂ is up to 200 picoseconds, more preferably up to 175 picoseconds, still more preferably up to 150 picoseconds, more preferably still up to 125 picoseconds, especially up to 100 picoseconds, more especially up to 75 picoseconds, still more especially up to 50 picoseconds, more especially still up to 30 picoseconds.

Preferably, ΔT₁₋₂ is from 2 picoseconds, more preferably from 5 picoseconds, still more preferably 7 picoseconds, more preferably still from 10 picoseconds, especially from 12 picoseconds, more especially from 15 picoseconds, still more especially from 18 picoseconds, more especially still from 20 picoseconds. Preferably, ΔT₁₋₂ is from 2 to 200 picoseconds, more preferably from 5 to 150 picoseconds, still more preferably from 10 to 100 picoseconds, more preferably still from 15 to 50 picoseconds, especially from 20 to 30 picoseconds, more especially from 22 to 28 picoseconds, still more especially from 24 to 27 picoseconds. In a preferred embodiment, the increment ΔT₁₋₂ is from 26 to 27 picoseconds, especially about 26.5 picoseconds.

Thereafter, the system operates to detect a reflection of the third pulse P₃ a time T₃seconds after the pulse P₃ has been emitted. The time period T₃ may have the values indicated above for the time period T₁, with the proviso that T₃ is longer than T₂. The time increment ΔT₂₋₃ between T₂ and T₃ may be any suitable period. Suitable values for the increment are as indicated above for ΔT₁₋₂. The reflected pulse corresponding to the emitted pulse P₃ will be a reflection from a surface that is further from the transmitter and receiver than both of the reflected pulses corresponding to pulses P₁ and P₂.

Thereafter, the operation of detecting a reflected pulse is repeated for each subsequent emitted pulse. Generally, for the mth pulse Pₘ of the plurality of pulses emitted by the transmitter, the system operates to detect a reflected pulse at time Tₘ seconds after the pulse Pₘ has been emitted. The time period Tₘ is longer than the time period Tₘ₋₁ for the preceding pulse Pₘ₋₁ by an increment of ΔT₍ₘ₋₁₎₋ₘ. Suitable values for each increment ΔT₍ₘ₋₁₎₋ₘ are as indicated above for ΔT₁₋₂.

The increments ΔT₍ₘ₋₁₎₋ₘ by which the time period Tₘ is increased for each successive emitted pulse Pₘ may be different. More preferably, the increments ΔT₍ₘ₋₁₎₋ₘ are the same for the plurality of pulses. A particularly preferred increment ΔT₍ₘ₋₁₎₋ₘ is 26.5 picoseconds.

The total number N of pulses in the train of pulses emitted by the transmitter may be any suitable number. Preferably, N is up to 2,000, more preferably up to 1,700, still more preferably up to 1,500, more preferably still up to 1,300, especially up to 1,200, more especially up to 1,000, still more especially up to 900, more especially still up to 800. In a preferred embodiment, N is up to 700, more preferably up to 600, still more preferably up to 550. Preferably, N is from 50, more preferably from 100, still more preferably from 150, more preferably still from 200, especially from 250, more especially from 300, still more especially from 350, more especially from 400. In a preferred embodiment, N is from 425, more preferably from 450, still more preferably from 475. Preferably, N is from 50 to 2,000, more preferably from 100 to 1,800, still more preferably from 150 to 1,500, more preferably still from 200 to 1,300, especially from 250 to 1,000, more especially from 300 to 800, still more especially from 350 to 700, more especially from 400 to 600. In a preferred embodiment, N is from 425 to 575, more preferably from 450 to 550, still more preferably from 475 to 525. In one preferred embodiment, N is from 500 to 520, more preferably from 510 to 515, especially 512.

In one preferred embodiment, the system and method of the present invention function to emitted a train of 512 pulses (N=512), a time period of 2 nanoseconds (T₁=2 nanoseconds) after the first pulse P₁ is emitted the system operates to search for a reflected pulse. Thereafter, 2 nanoseconds plus an increment of 26.5 picoseconds (ΔT₁₋₂=26.5 picoseconds; T₂=2.0265 nanoseconds) after the second pulse P₂ is emitted the system operates to search for a reflected pulse. Thereafter, for each successive pulse, the system waits a time Tₘ, where Tₘ is longer than Tₘ₋₁ by 26.5 picoseconds (ΔT₍ₘ₋₁₎₋ₘ=26.5 picoseconds; Tₘ=[2 nanoseconds+(m×26.5 picoseconds)]).

The product of the procedure applied to the N pulses in the train of pulses is a signal data set relating to the presence of objects and subjects in the region extending in front of the transmitter and receiver, from which pulses are reflected. This data set can be considered to be a frame of N consecutive measurements of reflected pulses, each corresponding to the features in the topography in the region scanned by the pulses. References herein to a ‘frame’ are to the set of signal data points generated from transmitting and detecting pulses from a single train of pulses.

Once each pulse in the train of N pulses has been emitted and the search for a corresponding reflected pulse after a time Tₘ has elapsed from emission, the procedure is preferably repeated.

It may be possible to identify the presence of a human subject using a single train of pulses. However, a single train of pulses may not provide sufficient data to accurately detect and identify the physiological signals associated with a human subject. Physiological signals can vary significantly between individual subjects and can be affected by various factors, for example age, health and overall physical condition. As a result, is especially preferred to repeat the aforementioned procedure and transmit a plurality of trains of pulses and detect reflected pulses as set out above.

The procedure may be repeated any suitable number of times necessary to allow movement patterns of the subject to be identified and monitored. In this way, a plurality of frames of signal data are generated, each providing a digital ‘image’ of the region being scanned over the time period of the train.

The transmission and detection of trains of pulses may be repeated at any suitable frequency. For example, the cycle of transmission and detection of the train of pulses may be repeated from 100 times per second, preferably from 200 times per second, more preferably from 300 times per second, more preferably still from 400 times per second, still more preferably from 500 times per second, especially from 600 times per second, more especially from 700 times per second, still more especially from 800 times per second.

In the specific embodiment described above for a train of 512 pulses (N=512), this procedure can be repeated about 900 times every second.

The detection of a reflected pulse indicates a surface in the region being scanned from which the emitted pulse has been reflected. The distance of the surface from the transmitter/receiver can be determined from the time Tₘ between the pulse being emitted and the reflected pulse being detected. By repeating the procedure of emitting the train of a plurality of N pulses and operating the receiver to detect any reflected pulses, a plurality of frames of the region being scanned is accumulated. Comparison of the reflected pulses, in particular their amplitude and their time of arrival, in different frames allows movement of the surface from which the pulses are being reflected to be identified. In general, subjects will exhibit movement, even if stood still, for example due to the action of breathing. Detecting movement allows the subjects to be distinguished from inanimate objects in the region being scanned. As described in more detail below, human subjects also generate a distinctive pattern of reflected pulses, which may be used to identify the presence of a subject in the region being scanned.

As described hereinbefore, the UWB radio wave pulses emitted by the transmitter are reflected back to the receiver by a surface. References herein to a ‘surface’ include both an external surface of a subject, for example the front of the torso of a subject when facing the transmitter, and an internal surface, for example the back of the torso of the subject when facing the transmitter.

In operation, the system and method of the present invention can be used to detect the movement of the chest wall of the subject, for example to detect the presence of the subject and/or monitor physiological signs of the subject, such as respiration rate and heart rate. With respect to heart rate, movement of the heart of the subject as it beats causes movement in the chest wall of the subject. It is this movement of the chest wall, corresponding to the action of the heart, that is readily detected by the system and method.

In operation of the system, the train of a plurality of UWB radio-wave pulses is emitted by the transmitter, under the control of the control unit. Suitable transmitters for emitting a train of UWB radio wave pulses are known in the art. The control unit further operates the receiver to identify any reflected pulses received at the appropriate time after each pulse has been emitted. The receiver generates a signal in response to a received reflected pulse. This signal is communicated to the processor of the control unit. The processor performs an analysis of the signals, as described in more detail hereinbelow.

As set out above, the detection of reflected pulses is carried out at timed intervals after the transmission of each pulse. The system may operate to switch the receiver on and off according to the timing of the detection of reflected pulses. In this way, the receiver provides batches of signal data to the processor corresponding to the periods of operation of the receiver to detect reflected pulses. Alternatively, in one preferred embodiment, the receiver is permanently on while the system is in operation. In this way, the receiver provides the processor with a continuous stream of signal data. The timed detection of the reflected pulses is carried out by the processor selecting batches of signal data received from the receiver and corresponding to the detection period appropriate for each transmitted pulse. In particular, the processor operates to receive a continuous stream of signal data from the receiver and select a batch of signal data for the duration of the detection period corresponding to the period beginning time Tₘ after the pulse Pₘ has been transmitted.

Each of the transmitter and the receiver comprise an antenna. The antenna used for the transmitter and the receiver may be the same or different. Suitable antennae configurations are known in the art. In one preferred embodiment, one or both antennae are a tapered slot antenna, for example a Vivaldi antenna or a linear tapered slot antenna. In an alternative embodiment, one or both of the antennae are a patch antenna.

Each antenna preferably has a frequency between 5 and 9 GHz, preferably between 6 and 8 GHz. The or each antenna is preferably centred in the aforementioned frequency ranges, more preferably having a frequency of from 6.5 to 7.5 GHz, still more preferably from 7 to 7.5 GHz. An antenna frequency of about 7.29 GHz has been found to be particularly suitable for many embodiments.

The system of the present invention preferably comprises a display. The display is operable to display data and information to a user under the control of the control unit. The display may be connected to the control unit by any suitable means, including a wired connection or, more preferably, a wireless connection. Suitable communication protocols for use with the display are known in the art and include WiFi and Bluetooth.

In addition to communicating with the display, the control unit may also communicate with a remote system or device, for example to transmit and record data regarding the operation of the system. Communication with the remote system or device may use any suitable protocol, such as WiFi and/or Bluetooth.

As discussed above, analysis of the signal data corresponding to the reflected pulses received by the receiver is carried out to identify the presence of a subject and, optionally to monitor their physiological signs, for example heartbeat and respiration rate. Analysis of the received reflected pulses includes such parameters as the strength or amplitude of the detected pulse and the timing of the received pulse relative to the time of the transmitted pulse.

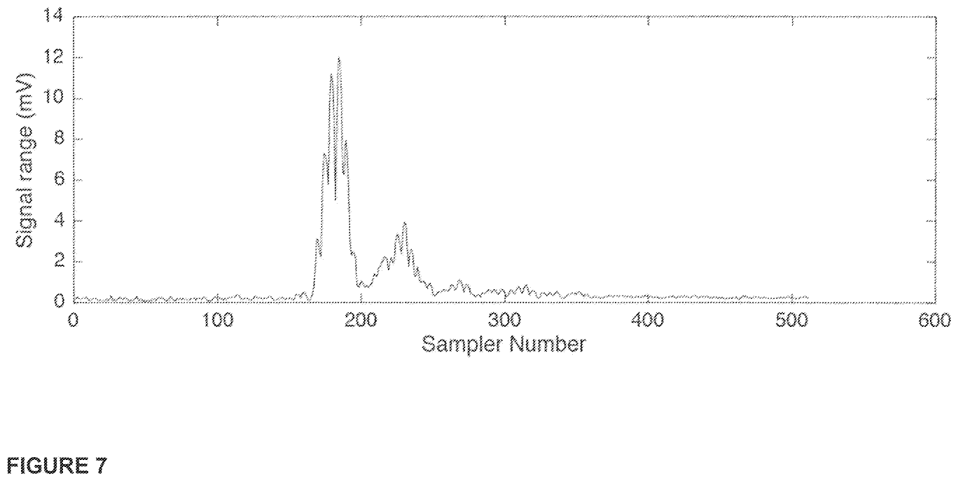

In the case of a human subject, a first, strong peak in the reflected pulses is generated at a first time when the transmitted pulses reach the subject and a second peak, weaker than the first peak, in the reflected pulses is generated at a later time. The timing of the second peak relative to the first peak corresponds to a distance of 15 cm, 25 cm, or 40 to 100 cm, depending upon the orientation of the subject relative to the transmitter. An example of this pattern of peaks is shown in FIG. 7 and described in more detail below. This pattern of two peaks and their relative strengths or amplitudes is a characteristic of a human subject and may be used to identify the presence of a human subject in the region being scanned. In particular, the processor can operate to analyse the signal data related to the reflected pulses and compare the data with a stored profile characteristic of the reflected pulses from a human subject. In this way, scanning the region of interest with a single train of a plurality of pulses and analysing the reflected pulses an increasing time increments, as described above, allows for the identification and location of a human subject.

In one preferred embodiment, the system and method of the present invention are used to detect and monitor the physiological signs of a subject. The physiological signs include heart rate and respiration rate. Other physiological signs include conditions or events which induce movements in part or all of the subject, for example movements arising when the subject coughs, has a seizure or other condition inducing movement in part or all of the subject.

By repeating the procedure described above and transmitting repeated trains of N pulses, with the corresponding detection of reflected pulses at incrementally increased times from transmission, the detected reflected pulses can be analysed and aspects of the physiological signs of the subject can be determined.

For example, in the case of determining the heart rate and/or respiration rate of the subject, the signal data from the receiver can be analysed in the processor using Fourier analysis, preferably Fast Fourier Transforms (FFT). FFT is a widely used analytical technique for computing the Discrete Fourier Transform (DFT) of a sequence of data. FFT is the preferred form of Fourier analysis for use in the present invention.

In the context of detecting heart rate, Fourier analysis, such as FFT, is applied to the signal data of the detected reflected pulses to obtain the frequency spectrum of the signal data and provide an indication of the heart rate and/or the respiration rate of the subject.

It has been found that the period of time, or ‘window’, over which the subject is repeatedly scanned using the procedure described above can affect the accuracy and resolution of the detected heart rate. The period of time over which the subject is scanned can be selected according to the required accuracy and resolution of the results. Preferably, when a determination of the heart rate of the subject is required, the subject is scanned for at least 1 second, more preferably at least 2 seconds, still more preferably at least 3 seconds, more preferably still at least 4 seconds, especially at least 5 seconds. The subject may be scanned for a longer period of time, as required. Throughout the period during which the subject is being monitored, the subject may be scanned at intervals for discrete periods of time. Alternatively, the subject may be scanned continuously during the monitoring period.

When scanning the subject for a window of 5 seconds, the subject is scanned with a plurality of trains of pulses and the reflected pulses detected as described above. The signal data corresponding to the detected reflected pulses is subjected to Fourier analysis, such as FFT analysis. The output of the Fourier analysis will exhibit peaks at the frequencies corresponding to the heart rate and its harmonics. By locating the highest peak in the frequency spectrum, an indication of the heart rate of the subject is obtained.

The advantage of using a 5-second window is that it provides a relatively accurate estimate of the heart rate within a short duration. However, some subtle changes in the heart rate that occur over a longer duration may be missed. Therefore, a longer scanning period is often preferable.

For example, when scanning the subject for a period of 10 seconds, the subject is scanned with a plurality of trains of pulses and the reflected pulses detected as described above. The signal data corresponding to the detected reflected pulses is subjected to Fourier analysis, such as FFT analysis. The output of the Fourier analysis will exhibit peaks at the frequencies corresponding to the heart rate and its harmonics. By locating the highest peak in the frequency spectrum, an indication of the heart rate of the subject is obtained, as is the case with a 5-second scan described above. However, the longer window size provides a more accurate estimate of the heart rate over a longer duration. This is particularly useful in detecting subtle changes in the heart rate, such as when the subject is exercising or is asleep.

Similarly, the techniques of the present invention can be employed to obtain an indication of the respiration rate of the subject and to monitor the respiration of the subject over a period of time. In particular, Fourier analysis can be applied to the signal data corresponding to the detected reflected pulses to obtain the frequency spectrum of the signal and thus detect the breathing rate.

As with the heart rate determination, the choice of sample window size, that is the length of time the subject is scanned, can affect the accuracy and resolution of the detected breathing rate. Preferably, when a determination of the respiration rate of the subject is required, the subject is scanned for at least 5 seconds, more preferably at least 10 seconds, still more preferably at least 15 seconds, more preferably still at least 20 seconds, especially at least 25 seconds. The subject may be scanned for a longer period of time, as required. Throughout the period during which the subject is being monitored, the subject may be scanned at intervals for discrete periods of time. Alternatively, the subject may be scanned continuously during the monitoring period.

For example, when scanning a subject over a period of 20, 40 and 80 seconds to monitor their respiratory function, the method functions as follows:

20-Second Sample Window Size: A 20-second segment of the signal data corresponding to the detected reflected pulses is subjected to Fourier analysis, such as FFT analysis, to compute its frequency spectrum. The output of the Fourier analysis will show peaks at the frequencies corresponding to the breathing rate and its harmonics. By locating the highest peak in the frequency spectrum, an indication of the breathing rate is obtained. The advantage of using a 20-second window is that it provides a relatively accurate estimate of the breathing rate over a moderate duration. However, using a 20-second sample window may miss some subtle changes in the breathing rate that occur over a longer duration.

40-Second Sample Window Size: In this embodiment, a 40-second segment of the signal data is subjected to Fourier analysis, such as FFT analysis, to compute its frequency spectrum. The output of the Fourier analysis will show peaks at the frequencies corresponding to the breathing rate of the subject and its harmonics, similar to the 20-second window case. However, the longer window size provides a more accurate estimate of the breathing rate over a longer duration. This is particularly useful in detecting subtle changes in the breathing rate, such as when the subject is exercising or is sleeping.

80-Second Sample Window Size: In this embodiment, an 80-second segment of the signal data is subjected to Fourier analysis, such as FFT analysis, to compute its frequency spectrum. The output of the Fourier analysis will show peaks at the frequencies corresponding to the breathing rate of the subject and its harmonics, similar to the 20-and 40-second window cases. However, the longer window size provides a more accurate estimate of the breathing rate over an even longer duration. This is particularly useful in detecting changes in the breathing rate that occur over an extended period, such as during prolonged sleep or relaxation.

The Fourier analysis is performed on the signal data of a plurality of frames collected over the period of the sample window. If the number n of sample data points is too low, the Fourier analysis may not provide an indication of heart rate or respiration rate of the subject. If this occurs, the number n of sample data points, that is the number of frames being analysed can be increased until the Fourier analysis provides an indicated heart or respiration rate. Preferably, n is at least 300, more preferably at least 350, still more preferably at least 400, more preferably still at least 450, especially at least 500. The maximum number of sample data points will be determined by the number of frames of signal data detected over the period of the sample window. n is preferably a power of 2, that is 2^{x}, where x is an integer, preferably wherein x is at least 8, more preferably at least 9, still more preferably at least 10, more preferably still at least 11. In some preferred embodiments x is 12.

The Fourier analysis, such as FFT analysis, is preferably performed on the signal data corresponding to the detected reflected pulses of the different sample window sizes according to the following protocol:

| 5-second | 10-second | 20-second | 40-second | 80-second |
|---|---|---|---|---|
| window | window | window | window | window |
| n = 256 | n = 256 | n = 256 | n = 256 | n = 256 |
| n = 512 | n = 512 | n = 512 | n = 512 | n = 512 |
| | n = 1024 | n = 1024 | n = 1024 | n = 1024 |
| | | n = 2048 | n = 2048 | n = 2048 |
| | | | n = 4096 | n = 4096 |

When analysing the signal data corresponding to the detected reflected pulses, it is preferable to analyse the reflected pulse data for each frame from the front of the subject to the back of the subject. In this way, the Fourier algorithm can more readily identify the respiration rate and the heart rate of the subject.

The signal data generated from the detected reflected pulses in each frame may be subjected to additional analysis before being subjected to Fourier analysis, such as FFT analysis.

The signal data for each frame may be passed to one or more FIFO buffers, each buffer preferably having a number of registers N equal to the number of pulses N emitted in each train of pulses. The number of frames of data correspond to the length of the sample window, for example 5, 10, 20, 40 or 80 seconds, as discussed above. Typically, the number of frames will range from 5,000 to 80,000 in this case.

The signal data for each frame may be analysed to calculate the first-in-first-out (FIFO) maximum, minimum and range. This analysis when applied to different window sizes provides an indication of the trade-off between frequency and time resolution, the reduction of spectral leakage, computational efficiency and the detection of low-frequency components. The results of this analysis can be used to select an appropriate window size for a specific application of the system and method, in turn improving the signal analysis. In particular, this allows for improved accuracy in extracting information from the train of pulses.

In addition, the signal data for each frame may be analysed to determine the full-width, half maximum (FWHM) for each peak of the signal data. This allows the processor to characterise the peaks of the signal data. In particular, by determining the signal threshold, peak detection, conducting FWHM measurements and determining the distance peak-to-peak, the processor is able to make a comparison between the signal data collected and the stored characteristics of a human subject. In this way, the processor is able to identify more readily when a human subject is being scanned and detected. This allows the portions of the signal data relating to the human subject to be selected for further processing. In particular, it is possible to select data relating to only those reflected pulses exhibiting peaks for further processing, for example by FFT analysis.

In addition, in a preferred embodiment, the signal data corresponding to the detected reflected pulses for each frame are subjected to a determination of the quality of the signal. This may be achieved by determining the area under the curve for some or all of the peaks and the total area under the curve for the entire set of signal data for that particular train of pulses.

In principle, it is possible to subject all the signal data corresponding to all the reflected pulses for each frame detected by the system to analysis, preferably Fourier analysis, in particular FFT analysis. However, it is more preferable to select only those signal data for each frame relating to the subject. Therefore, before Fourier analysis is conducted on the signal data, the signal data are preferably subjected to analysis as described above to identify those reflected pulses and the corresponding peaks in the signal data which relate to the subject. A selection of the signal data relating to the subject is then made. In addition, it is preferable to determine the quality of the peaks in the signal data, as described above. A selection of the signal data of sufficient quality is then made.

The thus selected signal data may then be analysed further, for example using FFT analysis, to determine characteristics of the movement pattern of the subject, such as movement associates with physiological signs, for example heart rate and respiratory rate. By making a selection of this kind, the Fourier analysis is applied to only those signal data points that are relevant to the determination being made and analysing irrelevant data points is avoided. This in turn increases the speed of the analysis, leading to an increased efficiency in the operation of the system and the method.

The signal data to be analysed for frequency components are preferably processed in a Fourier Transform Bank. Preferably, multiple filter blocks with varying time windows are applied to the signal data. This improves the reduction of noise in the signal data and improves the detection of non-stationary signals and enhanced time-frequency domain performance, which in turns results in more accurate processing of the signal data. Preferably, each filter block operates on a different time window to capture different frequency components of the signal data. The output of each filter block is a time-frequency representation of the input signal, which may be presented in the form of a spectrogram or scalogram.

Preferably, the time-frequency representations of the signal data obtained from the multiple filter blocks are averaged to provide an averaged time-frequency representation combining the information from all the different time windows. Peaks in the time-frequency representation can then be identified and then ranked. The highest ranked peaks may be selected for further analysis, as they represent the most significant frequency components of the signal data.

One particular application for monitoring the respiratory rate of a subject is situations where the subject has a condition that affects their normal respiratory function. One example is a subject suffering from sleep apnoea.

In a further aspect, the present invention provides a method for monitoring a subject to determine their heart rate and/or respiratory rate, the method comprising:

emitting a plurality of ultra-wide band (UWB) radio-wave pulses at the subject or an expected location of the subject;

detecting a reflected pulse for the first pulse of the plurality of pulses a time period T₁ seconds after the first pulse P₁ has been emitted;

detecting a reflected pulse for each subsequent emitted pulse Pₘ, where m is the number of the pulse, a time period Tₘ seconds after the pulse Pₘ has been emitted, wherein Tₘ is longer than the time period Tₘ₋₁ for the preceding pulse Pₘ₋₁;

generating signal data corresponding to each of the detected reflected pulses; and

analysing the signal data corresponding to the reflected pulses detected;

wherein the signal data are subjected to Fast Fourier Transform (FFT) analysis to determine the heart rate and/or the respiration rate of the subject.

In a still further aspect, the present invention provides a system for monitoring a subject to determine their heart rate and/or respiratory rate, the system comprising:

a transmitter operable to emit a plurality of ultra-wide band (UWB) radio-wave pulses at the subject or an expected location of the subject;

a receiver operable to:
detect a reflected pulse for the first pulse of the plurality of pulses a time period T₁ seconds after the first pulse P₁ has been emitted; and
detect a reflected pulse for each subsequent emitted pulse Pₘ, where m is the number of the pulse, a time period Tₘ seconds after the pulse Pₘ has been emitted, wherein Tₘ is longer than the time period Tₘ₋₁ for the preceding pulse Pₘ₋₁;

in use the receiver generating signal data corresponding to each of the detected reflected pulses; and

a processor for analysing the signal data corresponding to the reflected pulses detected;

wherein the signal data are subjected to Fast Fourier Transform (FFT) analysis to determine the heart rate and/or the respiration rate of the subject.

In one preferred embodiment, the system and method of the present invention are used to detect the presence of a subject, for example when the subject cannot be seen, such as when the subject is within a closed room. Other applications of this embodiment include detecting subjects in emergency situations, such as disaster areas, for example where a subject may be buried as a result of an earthquake, landslide or the like.

By repeating the procedure described above and transmitting repeated trains of N pulses, with the corresponding detection of reflected pulses at incrementally increased times from transmission, the detected reflected pulses can be analysed and movement of the subject can be detected. Movement that may be detected in this way include subjects walking and running. In addition, the presence of a subject may be detected when they are standing still, as a result of their breathing pattern.

Preferably the signal data from the receiver are analysed in the processor using Fourier analysis, preferably Fast Fourier Transforms (FFT). As noted above, FFT is a widely used analytical technique for computing the Discrete Fourier Transform (DFT) of a sequence of data. In the context of detecting movement of a subject, FFT is applied to the signal data of the detected reflected pulses to obtain the frequency spectrum of the signal data, in turn indicating movement of the subject.

It has been found that the period of time over which the subject is repeatedly scanned using the procedure described above can affect the accuracy and resolution of the indication of the detected movement. The period of time over which the subject is scanned can be selected according to the required accuracy and resolution of the results. Preferably, the subject is scanned for at least 1 second, more preferably at least 2 seconds, still more preferably at least 3 seconds, more preferably still at least 4 seconds, especially at least 5 seconds. The subject may be scanned for a longer period of time, as required, for example up to 10 seconds, 15 seconds or longer, as required to obtain an indication of movement and the presence of a subject. Throughout the period during which the subject is being monitored, the subject may be scanned at intervals for discrete periods of time. Alternatively, the subject may be scanned continuously during the monitoring period.

When scanning the subject for a period of time, the subject is scanned with a plurality of trains of pulses and the reflected pulses detected as described above. The signal data corresponding to the detected reflected pulses are subjected to Fourier analysis, for example FFT analysis. The output of the Fourier analysis will exhibit peaks at the frequencies corresponding to the movement pattern of the subject and its harmonics.

The Fourier analysis is performed on the signal data of a plurality of frames collected over the period of the sample window. If the number n of sample data points is too low, the Fourier analysis may not provide an indication of movement of the subject. If this occurs, the number n of sample data points, that is the number of frames being analysed can be increased until the Fourier analysis provides an indicated heart or respiration rate. Preferably, n is at least 300, more preferably at least 350, still more preferably at least 400, more preferably still at least 450, especially at least 500. The maximum number of sample data points will be determined by the number of frames of signal data detected over the period of the sample window. n is preferably a power of 2, that is 2x, where x is an integer, preferably wherein x is at least 8, more preferably at least 9, still more preferably at least 10, more preferably still at least 11. In some preferred embodiments x is 12.

The Fourier analysis, preferably FFT analysis, is preferably performed on the signal data corresponding to the detected reflected pulses of the different sample window sizes according to the following protocol:

| 5-second | 10-second | 20-second | 40-second | 80-second |
|---|---|---|---|---|
| window | window | window | window | window |
| n = 256 | n = 256 | n = 256 | n = 256 | n = 256 |
| n = 512 | n = 512 | n = 512 | n = 512 | n = 512 |
| | n = 1024 | n = 1024 | n = 1024 | n = 1024 |
| | | n = 2048 | n = 2048 | n = 2048 |
| | | | n = 4096 | n = 4096 |

The signal data generated from the detected reflected pulses in each train of pulses may be subjected to additional analysis before being subjected to Fourier analysis, such as FFT analysis.

The signal data for each train of pulses may be passed to one or more FIFO buffers, each buffer preferably having a number of registers N equal to the number of pulses N emitted in each train of pulses. The number of frames of data correspond to the length of the sample window, for example 5, 10, 20, 40 or 80 seconds, as discussed above. Typically, the number of frames will range from 5,000 to 80,000 in this case.

The signal data for each train of pulses may be analysed to calculate the first-in-first-out (FIFO) maximum, minimum and range. This analysis when applied to different window sizes provides an indication of the trade-off between frequency and time resolution, the reduction of spectral leakage, computational efficiency and the detection of low-frequency components. The results of this analysis can be used to select an appropriate window size for a specific application of the system and method, in turn improving the signal analysis. In particular, this allows for improved accuracy in extracting information from the train of pulses.

In addition, the signal data for each frame may be analysed to determine the full-width, half maximum (FWHM) for each peak of the signal data. This allows the processor to characterise the peaks of the signal data. In particular, by determining the signal threshold, peak detection, conducting FWHM measurements and determining the distance peak-to-peak, the processor is able to make a comparison between the signal data collected and the stored characteristics of a human subject. In this way, the processor is able to identify more readily when a human subject is being scanned and detected. This allows the portions of the signal data relating to the human subject to be selected for further processing. In particular, it is possible to select data relating to only those reflected pulses exhibiting peaks for further processing, for example by FFT analysis.

In addition, in a preferred embodiment, the signal data corresponding to the detected reflected pulses for each frame are subjected to a determination of the quality of the signal. This may be achieved by determining the area under the curve for some or all of the peaks and the total area under the curve for the entire set of signal data for that particular train of pulses.

In principle, it is possible to subject all the signal data corresponding to all the reflected pulses for each frame detected by the system to analysis, in particular FFT analysis. However, it is more preferable to select only those signal data for each frame relating to the subject. Therefore, before Fourier analysis is conducted on the signal data, the signal data are preferably subjected to analysis as described above to identify those reflected pulses and the corresponding peaks in the signal data which relate to the subject. A selection of the signal data relating to the subject is then made. In addition, it is preferable to determine the quality of the peaks in the signal data, as described above. A selection of the signal data of sufficient quality is then made. The thus selected signal data may then be analysed further, for example using FFT analysis, to determine characteristics of the movement pattern of the subject. As noted above, by making a selection of this kind, the Fourier analysis, such as FFT analysis, is applied to only those signal data points that are relevant to the determination being made and analysing irrelevant data points is avoided. This in turn increases the speed of the analysis, leading to an increased efficiency in the operation of the system and the method.

The signal data to be analysed for frequency components are preferably processed in a Fourier Transform Bank. Preferably, multiple filter blocks with varying time windows are applied to the signal data. This improves the reduction of noise in the signal data and improves the detection of non-stationary signals and enhanced time-frequency domain performance, which in turns results in more accurate processing of the signal data. Preferably, each filter block operates on a different time window to capture different frequency components of the signal data. The output of each filter block is a time-frequency representation of the input signal, which may be presented in the form of a spectrogram or scalogram.

Preferably, the time-frequency representations of the signal data obtained from the multiple filter blocks are averaged to provide an averaged time-frequency representation combining the information from all the different time windows. Peaks in the time-frequency representation can then be identified and then ranked. The highest ranked peaks may be selected for further analysis, as they represent the most significant frequency components of the signal data.

In a further aspect, the present invention provides a method for detecting the movement of a subject, the method comprising:

emitting a plurality of ultra-wide band (UWB) radio-wave pulses at the subject or an expected location of the subject;

detecting a reflected pulse for the first pulse of the plurality of pulses a time period T₁ seconds after the first pulse P₁ has been emitted;

detecting a reflected pulse for each subsequent emitted pulse Pₘ, where m is the number of the pulse, a time period Tₘ seconds after the pulse Pₘ has been emitted, wherein Tₘ is longer than the time period Tₘ₋₁ for the preceding pulse Pₘ₋₁;

generating signal data corresponding to each of the detected reflected pulses; and

analysing the signal data corresponding to the reflected pulses detected;

wherein the signal data are subject to Fast Fourier Transform (FFT) analysis to identify movement of the subject.

In a still further aspect, the present invention provides a system for detecting the movement of a subject, the system comprising:

a transmitter operable to emit a plurality of ultra-wide band (UWB) radio-wave pulses at the subject or an expected location of the subject;

a receiver operable to:

detect a reflected pulse for the first pulse of the plurality of pulses a time period T₁ seconds after the first pulse P₁ has been emitted; and

detect a reflected pulse for each subsequent emitted pulse Pₘ, where m is the number of the pulse, a time period Tₘ seconds after the pulse Pₘ has been emitted, wherein Tₘ is longer than the time period Tₘ₋₁ for the preceding pulse Pₘ₋₁;

in use the receiver generating signal data corresponding to each of the detected reflected pulses; and

a processor operable to analyse the signal data corresponding to the reflected pulses detected;

wherein the signal data are subjected to Fast Fourier Transform (FFT) analysis to identify movement of the subject.

Embodiments of the present invention will now be described, by way of example only, having reference to the accompanying drawings, in which:

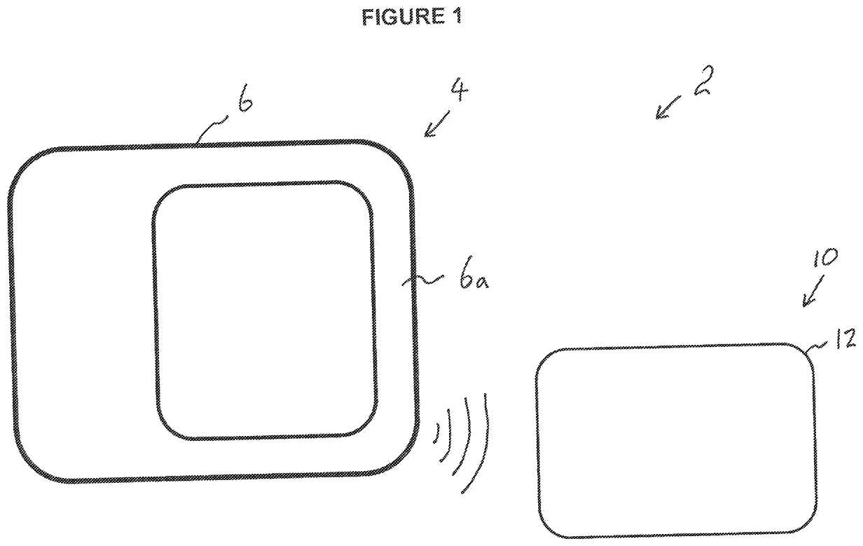

FIG. 1 is a diagrammatical representation of a system according to one embodiment of the present invention;

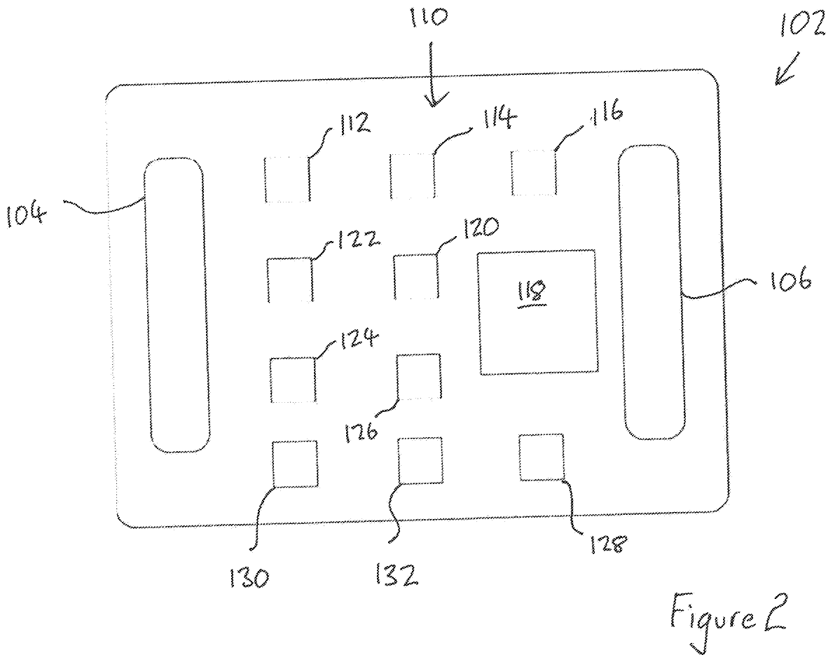

FIG. 2 is a top-level diagrammatical representation of one embodiment of a printed circuit board (PCB) for use in the system of the present invention;

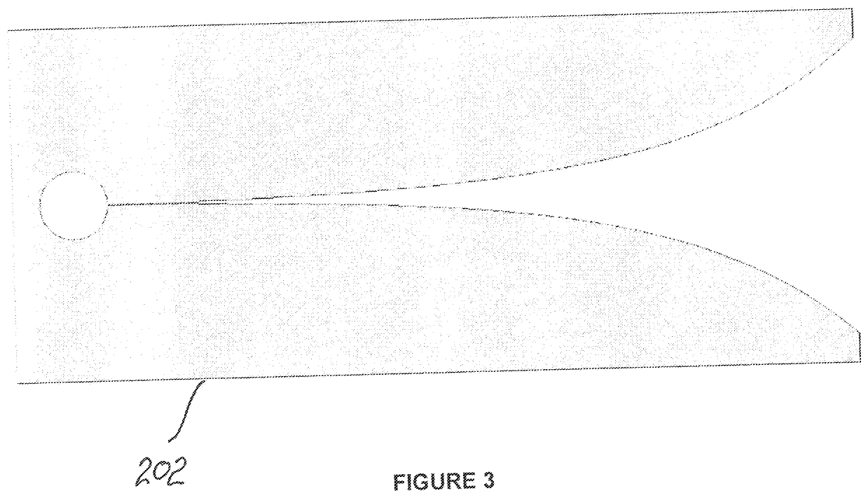

FIG. 3 is a representation of one embodiment of an antenna for use in the system of the present invention;

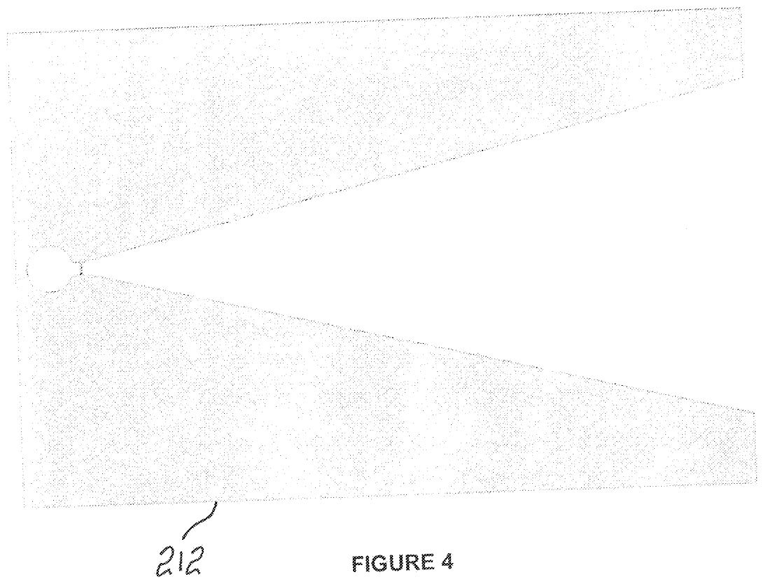

FIG. 4 is a representation of an alternative embodiment of an antenna for use in the system of the present invention;

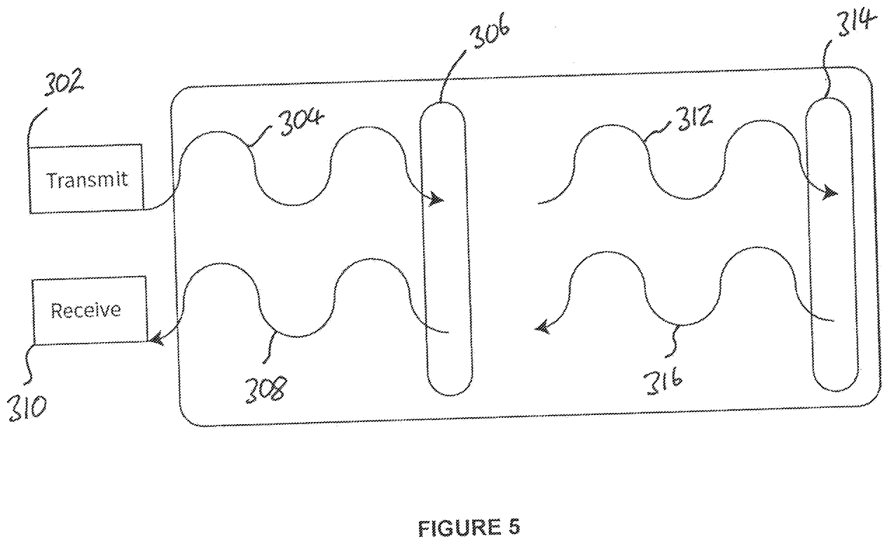

FIG. 5 is a diagrammatical representation of the transmission and reception of pulses during the method of the present invention;

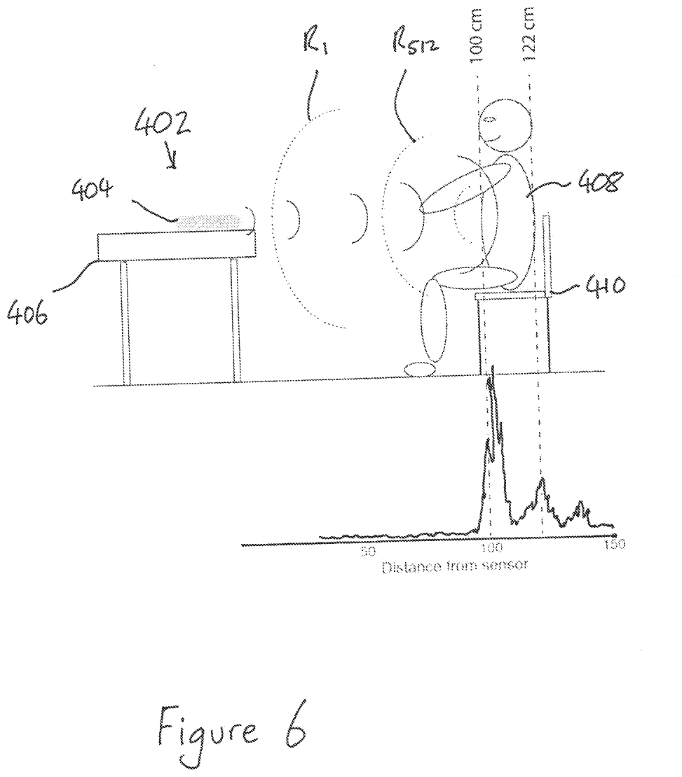

FIG. 6 is a diagrammatical representation of one embodiment of the method of the present invention in use to monitor the physiological signs of a subject;

FIG. 7 is a graphical representation of signal data received for one train of pulses of the method of FIG. 6;

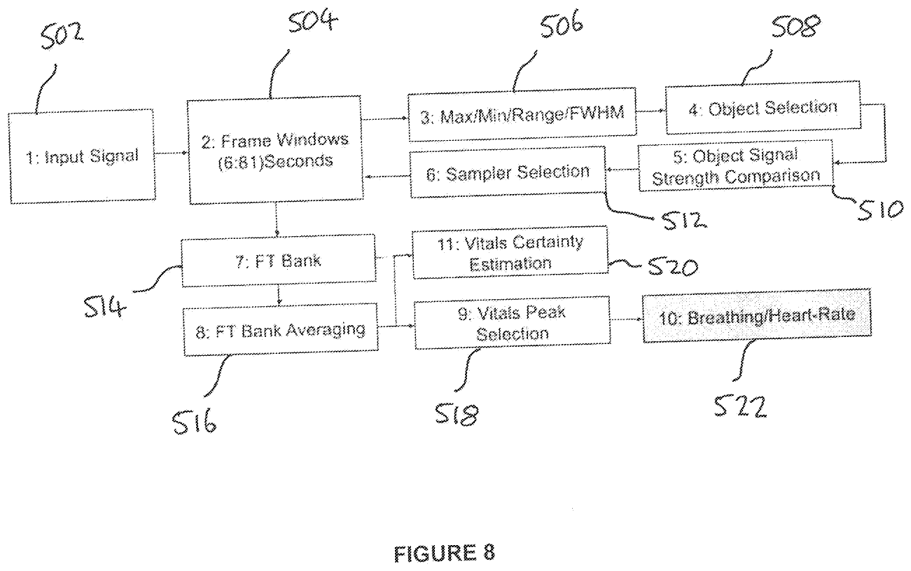

FIG. 8 is a diagrammatical representation of one embodiment of the signal processing procedure for determining the respiration rate and heart rate of a subject;

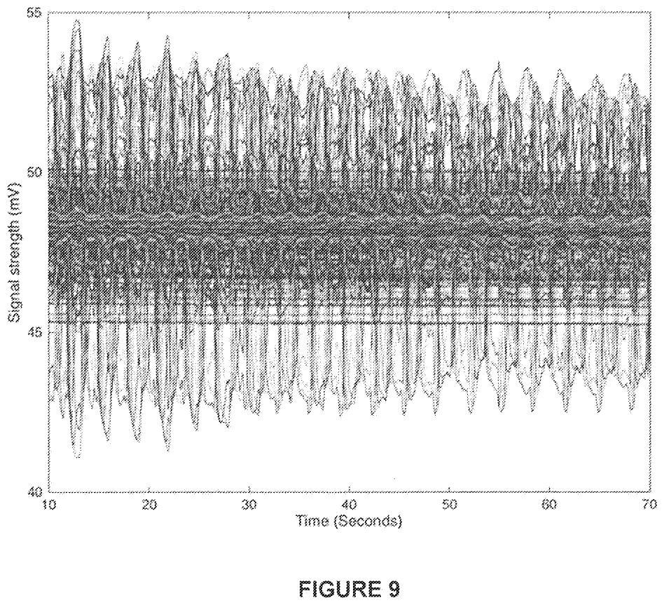

FIG. 9 is a graph showing the signal data generated from sampling reflected pulses from a subject in one embodiment of the present invention;

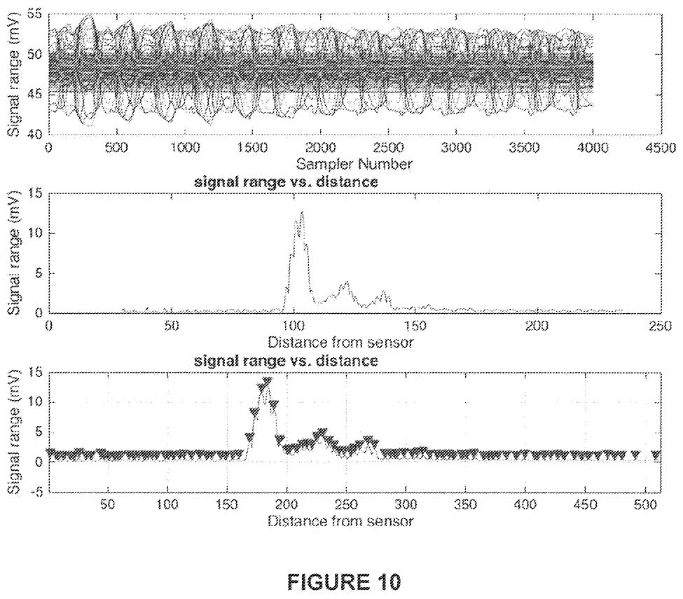

FIG. 10 is graphs showing the signal data of FIG. 9 after being processed in a first selection stage;

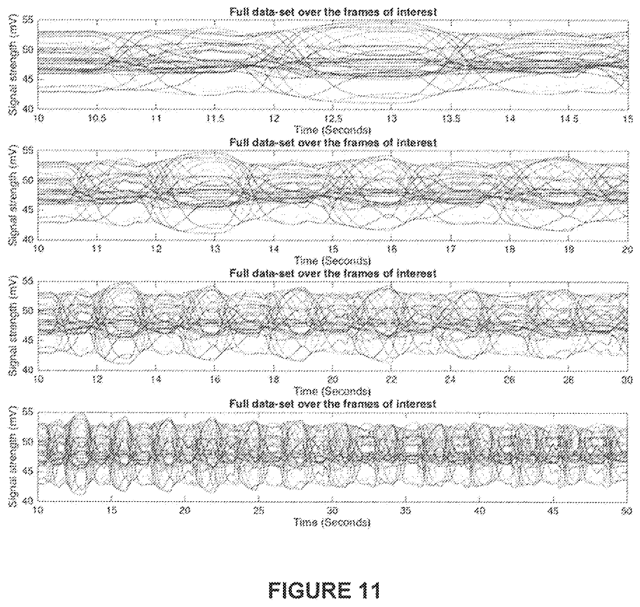

FIG. 11 is a graph showing the full subject-related signal data set selected from the data shown in FIG. 10;

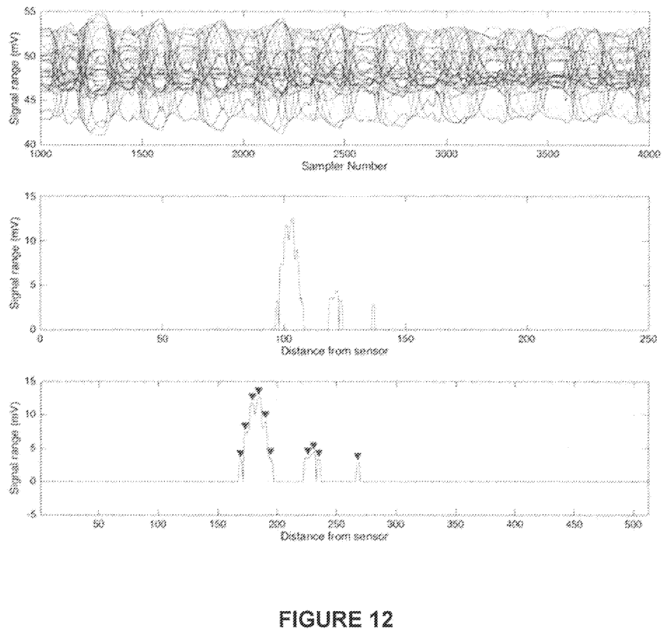

FIG. 12 is graphs showing the subject-related signal peaks selected from the full data set shown in FIG. 11;

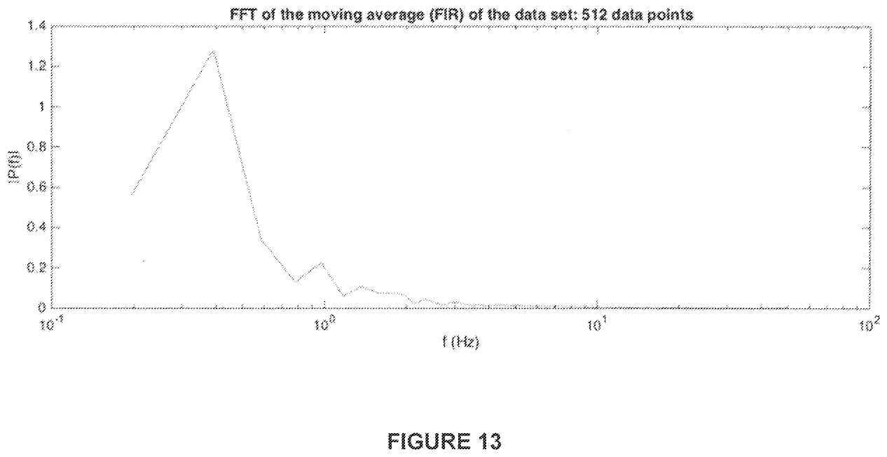

FIG. 13 is a graph showing the results of the FFT analysis of the selected data set shown in FIG. 12 using 512 data points;

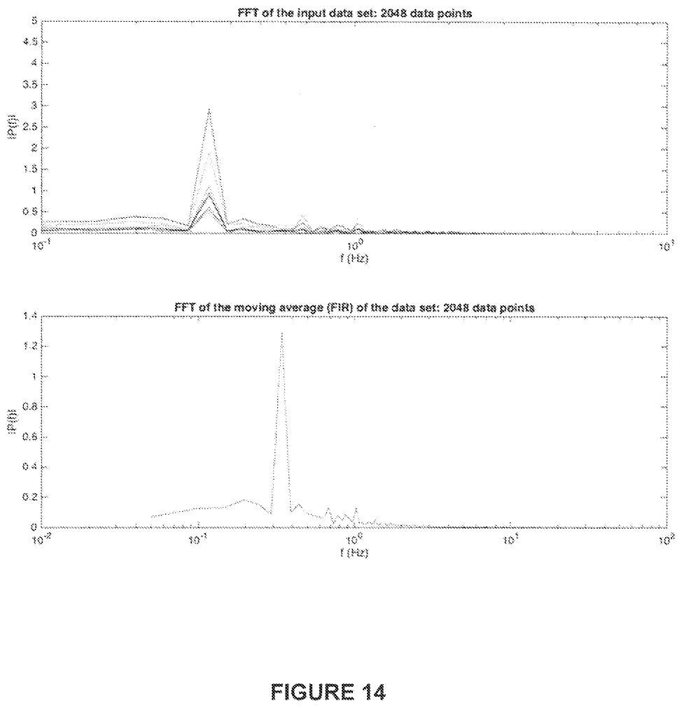

FIG. 14 is a graph showing the results of the FFT analysis of the selected data set shown in FIG. 12 using 1024 data points;

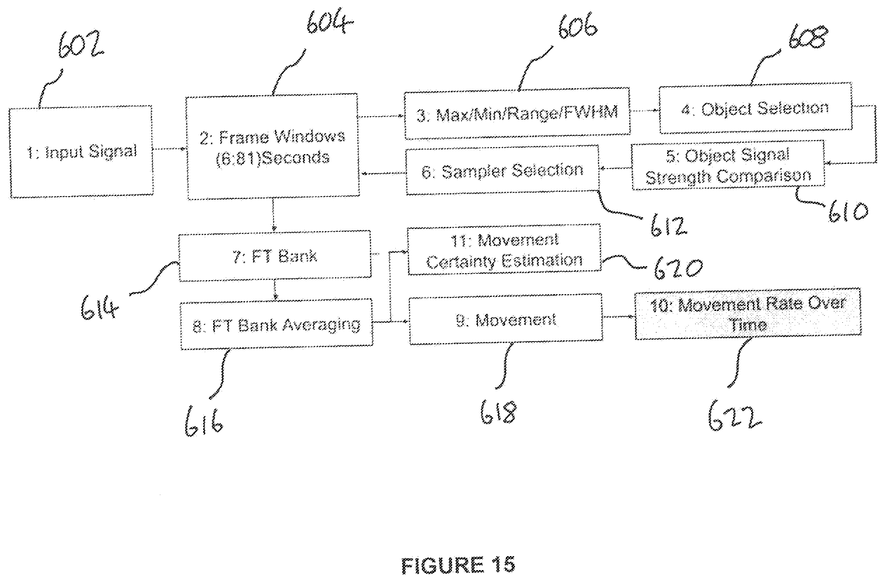

FIG. 15 is a diagrammatical representation of one embodiment of the signal processing procedure for determining the movement of a subject; and

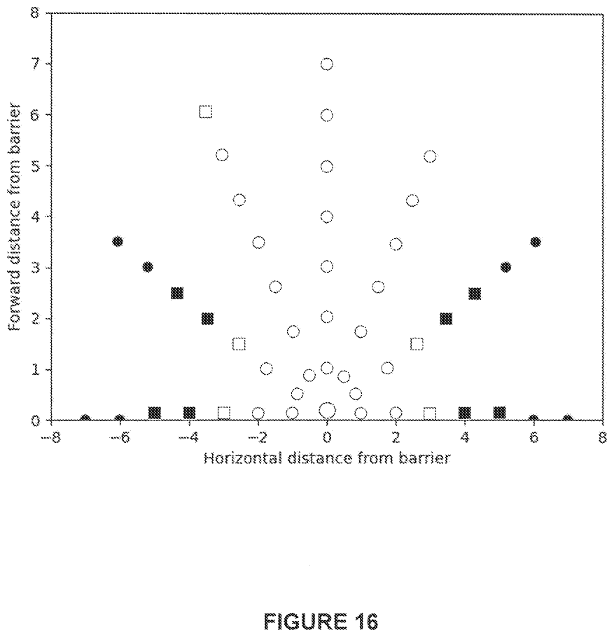

FIG. 16 is a graph showing the results of one embodiment of the system and method of the present invention detecting a person within a room.

Referring first to FIG. 1, there is shown a system according to one embodiment of the present invention. The system, generally indicated as 2, comprises a hand-held sensor device 4. The hand-held sensor 4 comprises a housing 6 having a handle portion 6a and containing components of the system, including a transmitter, a receiver, antennae, a processor for controlling the operation of the transmitter and the receiver and for processing signal data received from the receiver in response to detected reflected pulses, and a battery for providing electrical power to the components. The system shown in FIG. 1 further comprises a display 10 comprising a screen 12. The device 4 communicates with the display 10 by a wireless connection, for example a WiFi or Bluetooth wireless link.

Turning to FIG. 2, there is shown a top-level diagrammatical representation of one embodiment of a printed circuit board (PCB) for use in the system of the present invention. The components arranged on the PCB, generally indicated as 102, comprise:

A transmitter and antenna 104, from which trains of N UWB radio-wave pulses are emitted. Examples of configurations for the antenna of the transmitter 104 are shown in FIGS. 3 and 4 and described below.

A receiver and antenna 106 for detecting reflected pulses. Examples of configurations for the antenna of the receiver 106 are shown in FIGS. 3 and 4 and described below.

A control unit 110 comprising:

A controller 112 for controlling the transmission of the pulses and the detection of reflected pulses.

A data processor 114 for analysing and processing signal data received from the receiver 106.

A communication/display processor 116 for controlling communication of the device 2 with other devices and for compiling and displaying data on the display 10.

A battery 118, together with a battery charging module 120 and a power supply regulator 122.

A first communication module 124 for communication between the device 2 and a local area network (LAN).

A second communication module 126 for communication between the device 2 and remote devices by WiFi.

A third communication module 128 for communication between the device 2 and remote devices by Bluetooth.

Further components on the PCB 102 are an I2C controller 130 and an internal navigation system (INS) module 132.

Turning to FIG. 3, there is shown one embodiment of an antenna for use in the system of the present invention. The antenna, generally indicated as 202, may be used for one or both of the transmitter and receiver. The antenna 202 is co-planar antenna of the tapered slot or Vivaldi configuration, as is known in the art. The antenna 202 is tuned to a frequency of 7.29 GHz.

Turning to FIG. 4, there is shown an alternative embodiment of an antenna for use in the system of the present invention. The antenna, generally indicated as 212, may be used for one or both of the transmitter and receiver. The antenna 212 is a linear tapered slot, co-planar antenna. The antenna 212 is tuned to a frequency of 7.29 GHz.

Referring now to FIG. 5, there is shown a diagrammatical representation of the transmission and reception of pulses as occurs in the method of the present invention. In particular, the transmitter 302 emits a first pulse 304 of UWB radio waves. The first pulse 304 is reflected from a first surface 306, generating a first reflected pulse 308, which is received by a receiver 310 at time Tₐ seconds after the first pulse 304 is emitted. The transmitter 302 emits a second pulse 312, which is reflected from a second surface 314, generating a second reflected pulse 316, which is received by the receiver 310 at a time T_{b} seconds after the second pulse 312 is emitted. Time T_{b} is longer than time Tₐ, meaning the detection of the second reflected pulse 316 is delayed to allow the second reflected pulse 316 to be detected. By operating the receiver 310 at time Tₐ seconds after the first pulse 304 has been emitted, the first reflected pulse 308 is detected, indicating the presence of the first surface 306 at a first distance from the transceiver. Similarly, operating the receiver 310 at time T_{b} seconds after the first pulse 304 has been emitted, the first reflected pulse 308 is detected, indicating the presence of the first surface 306, at a second distance from the transceiver, greater than the first distance. In this way, a frame characterising the region being scanned by the pulses may be constructed using the detected reflected signals.

Referring to FIG. 6, there is shown a representation of a system of one embodiment of the present invention in use to monitor a subject. The system, generally indicated as 402, is contained within a single housing 404, located on a table 406, in front of which the subject 408 is seated on a chair 410.

In an embodiment of the present invention, Embodiment A, the system 402 is configured to generate repeated trains of pulses, each train comprising 512 UWB radio-wave pulses (N=512) having a frequency between 6 and 8 GHz. A pulse is transmitted every 20 nanoseconds. Following the transmission of the first pulse P₁, the receiver is activated after a delay T₁ of 2 nanoseconds and operates to detect whether a reflected pulse is received. The receiver is activated after each successive pulse, T₂, T₃, T₄. . . . Tₘ₋₁, Tₘ. . . . T₅₁₂, the delay in activating the receiver after the pulse transmission being increased incrementally by 26.5 picoseconds (that is ΔT₍ₘ₋₁₎₋ₘ is 26.5 picoseconds for all 512 pulses) up to a time T₅₁₂ of 15.6 nanoseconds.

This cycle of transmitting and receiving pulses is repeated up to 900 times per second. This generates up to 900 frames per second of signal data.

In FIG. 6, the first transmitted pulse P₁ and the last transmitted pulse P₅₁₂ are indicated as having generated two reflected pulses R₁ and R₅₁₂ indicated in the figure, these being the pulses reflected from the front (chest) of the subject 408 and the rear (back) of the subject respectively.

The strength of the signal data generated by the receiver in response to the reflected pulses detected by the system 402 is shown graphically in the lower part of FIG. 6. As can be seen, strong peaks are exhibited in the signal data at a distance of 100 cm from the system 402, corresponding to the chest of the subject 408, and a distance 122 cm from the system 402, corresponding to the back of the subject. This set of signals having the two aforementioned major peaks is characteristic of a human subject.

The signal strength of all 512 detected reflected pulses of one train of pulses, that is one ‘frame’, is shown graphically in FIG. 7. As can be seen, the signal data exhibit major peaks for pulses 180 to 200 and 220 to 250, corresponding to the chest and back of the subject 408.

Turning to FIG. 8, there is shown a diagrammatical representation of one embodiment of the signal processing algorithm for processing signal data, such as the data generated from the embodiment shown in FIG. 6, in particular for determining the heart rate and respiration rate of the subject.

The input to the process are the signal data 502 received from the receiver of the system corresponding to detected reflected pulses of each frame. In the case of Embodiment A of the system and method, the signal data 502 are transmitted to the processor via a USB2 port. The signal data has a frame rate of 100 MHz, each frame comprising 512 samples of the reflected pulses, each sample having a 16 bit signal. Therefore, input signal data are transmitted at a rate of less than 1 Mbit/second.

The signal data 502 are passed to 6 first-in-first-out (FIFO) buffers 504, each comprising 512 registers. The registers have data sizes corresponding to the length of the sample window. Thus, for sample windows of 5, 10, 20, 40 and 80 seconds, each register contains from 5,000 to 80,000 frames.

From the buffers 504, the signal data are subjected to a selection process, in which the peak signals relating to the subject being monitored of each frame are identified and selected. The selection process is as follows:

In a first selection stage 506, the signal data are analysed to determine the FIFO maximum, minimum and range of the signal data in each frame. To characterise the peaks in the detected pulses, the signal data are also subjected to full-width, half-maximum (FWHM) determinations.

There follows a subject selection stage 508. In operation, the system will detect a range of reflected pulses, some of which emanate from the subject of interest or being monitored, while others are reflected from other objects in the vicinity of the subject. To select the signal data relating to the subject, the distance of the object corresponding to the reflected pulses is determined. In addition the maximum signal strength of neighbouring peaks is determined. A human subject has a characteristic pattern of reflected pulses. In particular, a human subject may be identified by a strong first peak reflected pulse, corresponding to the front of the subject, that is the part of the subject closest to the transceiver, and a second, weaker peak reflected pulse emanating from some distance beyond the source of the first reflected peak. This distance is typically between 15, 25, or 40 cm to 100 cm, depending upon the orientation of the subject relative to the transceiver. A typical pattern of peaks for a human subject is shown in FIGS. 6 and 7, as discussed above. By determining the signal thresholds, detecting the peak signals, making FWHM determinations of the peaks, as well as determining the distance corresponding to the time between the peaks and the relative signal range provides a characterisation of the object from which the reflected pulses emanate. This characterisation may be compared with a stored characterisation of a human subject, allowing those peaks in the signal data corresponding to a human subject to be identified and selected.

The quality of the signal is determined in the next selection step 510. The signal quality is determined by measuring the area under the curves of the signal peaks corresponding to a human subject and the total area under the curve for the entire set of data for each frame.

Finally, signal selection 512 is performed, selecting only those peak signals from each frame that correspond to a human subject. The selected signal data are then subjected to analysis to determine the heart rate and/or respiration rate of the subject. This is achieved using Fourier analysis, in particular Fast Fourier Transform (FFT) analysis.

FFT analysis is performed in a Fourier Transform (FT) Bank 514. The signal data of each frame are sampled in the order corresponding to the direction of from the front of the subject to the back of the subject. The 5 and 10 second windows are utilised for heart rate detection and the 20, 40 and 80 second windows are used principally for respiration rate. An estimate of the respiration rate can be obtained by using signal data from just a 10 second window. FFT analysis is applied to the selected signal data on the basis of the following criteria, where ‘n’ is the number of frames in the data sample being analysed:

| 5-second | 10-second | 20-second | 40-second | 80-second |
|---|---|---|---|---|
| window | window | window | window | window |
| n = 256 | n = 256 | n = 256 | n = 256 | n = 256 |
| n = 512 | n = 512 | n = 512 | n = 512 | n = 512 |
| | n = 1024 | n = 1024 | n = 1024 | n = 1024 |
| | | n = 2048 | n = 2048 | n = 2048 |
| | | | n = 4096 | n = 4096 |

The signal data to be analysed for frequency components are processed in the FT Bank, as noted above. Multiple filter blocks with varying time windows are applied to the incoming signal data. This improves the reduction of noise in the signal data and improves the detection of non-stationary signals and enhanced time-frequency domain performance, which in turns results in more accurate processing of the signal data. Each filter block of the multiple filter blocks operates on a different time window to capture different frequency components of the signal data. The output of each filter block is a time-frequency representation of the input signal, which may be presented in the form of a spectrogram or scalogram.

All signal data processed in the FT Bank 514 are subjected to FT Bank Averaging 516, in which the signal data are averaged. The averaged signal data are used to estimate the certainty of the final determination and in peak selection. In particular, the time-frequency representations of the signal data obtained from the multiple filter blocks are averaged to provide an averaged time-frequency representation combining the information from all the different time windows. Averaging is performed by summing the values at each corresponding time-frequency point of the output of the FTT analysis across all the filter blocks and dividing the sum by the total number of filter blocks. This provides an averaged time-frequency representation that combines the information from all the time windows.

Peaks in the time-frequency representation are then be identified and then ranked. Peaks in the time-frequency representation represent the most prominent frequency components of the input signal data. The peaks are identified by searching for local maxima in the representation, that is points where the value is higher than both neighbouring point in both the time and frequency dimensions. The peaks thus identified are then ranked based on their magnitude. The magnitude of the peak represent the energy or power of the corresponding frequency component. The highest ranked peaks are selected for further analysis, as they represent the most significant frequency components of the signal data.

Thereafter, peak selection 518 is carried out. Peak selection is required to distinguish those peaks corresponding to the movement of the heart of the subject to be distinguished from the peaks corresponding to movement due to respiration of the subject. The data from the FFT analysis and averaging are analysed to identify the largest maximum peaks and the second largest maximum peaks. The largest maximum peaks in each frame correspond to the respiration movement and indicate the respiration rate, while the second largest maximum peaks correspond to movements arising from the heart of the subject beating.

The selected maximum peaks are then used to determine the heart rate and the respiration rate. The determination 518 is carried out using the largest maximum peaks to determine the respiration rate of the subject. These correspond to movements at the front and back of the body of the subject. The second largest maximum peaks correspond to the heart rate of the subject and are indicated by pulses emanating from a point in the torso of the subject several centimetres in from the chest wall.

A certainty estimation 520 is also performed by calculating the log FWHM of the maximum peaks corresponding to each of the respiration rate and heart rate, divided by the total energy spectra of the sample set. The total energy spectra of a sample set represents the distribution of energy across the different frequency components in the signal data. The total energy spectra provides an understanding of the contribution of each frequency component to the overall energy of the signal, in particular the energy distribution across different frequency components in a signal data. This is useful for identifying dominant frequency components, detecting changes in signal characteristics, and comparing the energy distribution of different signals. The total energy spectra is calculated as follows:
a) Convert the time-domain signal to the frequency domain: To analyze the energy distribution across different frequencies, the signal data are first transformed from the time domain to the frequency domain. This is achieved using the Fast Fourier Transform (FFT).
b) Calculate the magnitude spectrum: After obtaining the frequency-domain representation of the signal, the magnitude of each frequency component is calculated. The magnitude spectrum is the absolute value of each frequency component in the transformed signal.
c) Compute the power spectrum: The power spectrum represents the energy of each frequency component in the signal. To compute the power spectrum, the magnitude spectrum is squared.
d) Calculate the total energy: To find the total energy of the sample set, the power spectrum values are summed across all frequency components. This represents the overall energy of the signal. Optionally normalise the energy spectra: In some cases, it may be useful to normalise the energy spectra to better compare the energy distribution of different signals or to account for differences in signal length. To normalise the energy spectra, each power spectrum value is divided by the total energy.

The output 522 of the process is an indication for each of the heart rate and respiration rate of the subject. These indications may be displayed to the user, for example by way of a display as shown in FIG. 1. Alternatively or in addition, these indications may be stored within the system and/or transmitted to a remote device, such as a patient medical record system.

The system and method of the present invention when applied to monitoring the heart rate and respiration rate of a subject are further explained by way of the following working example, which is provided for illustrative purposes only.

### Example 1: Heart Rate and Respiration Rate Monitoring

The system 402 was configured to operate according to Embodiment A described above.

The system 402 was configured to generate repeated trains of pulses, each train comprising 512 UWB radio-wave pulses (N=512) having a frequency between 6 and 8 GHz. A pulse was transmitted every 20 nanoseconds. Following the transmission of the first pulse P₁, the receiver was activated after a delay T₁ of 2 nanoseconds and operated to detect whether a reflected pulse is received. The receiver was activated after each successive pulse, T₂, T₃, T₄ . . . Tₘ₋₁, Tₘ T₅₁₂, the delay in activating the receiver after the pulse transmission being increased incrementally by 26.5 picoseconds (that is ΔT₍ₘ₋₁₎₋ₘ is 26.5 picoseconds for all 512 pulses) up to a time T₅₁₂ of 15.6 nanoseconds.

This cycle of transmitting and receiving pulses was repeated up to 900 times per second, generating up to 900 frames per second of signal data.

The arrangement for monitoring the subject was as shown in FIG. 6 and described above. The system was operated for a period of 70 seconds.

Operating the system according to Embodiment A above produced a plurality of frames of signal data, each frame comprising 512 sampled reflected pulses. A combined plot of the signal data from the receiver corresponding to all 512 sampled reflected pulses for all frames generated during the 70 second operating period is shown in FIG. 9.

The signal data represented in FIG. 9 were input into a processor configured to perform the analysis summarised in FIG. 8 and described above.

The processed signal data produced from the first selection stage 506 are shown in FIG. 10. The upper graph of FIG. 10 shows the selected signal data points for all 512 frames of the set of data. As can be seen, the first selection stage 506 has detected the peaks in the signal data, as indicated in the lower two graphs of FIG. 10.

The processed signal data represented in FIG. 10 were further analysed in the subject selection stage 508 to identify those peaks related to the subject. Following the subject selection stage 508, the peaks identified in the first selection stage 506 are reduced to only those peaks relating to the subject being monitored, the signal data relating to objects other than the subject having been removed. The full selected subject-related signal data are shown in FIG. 11.

The signal data represented in FIG. 11 contains all the signal data relating to reflected pulses received from the subject in each frame. However, for the FFT analysis, it was necessary only to analyse the peak signals, in order to generate an indication of the heart rate and respiration rate of the subject. Selection of the peak signals from the full subject-related data set reduced the signal data in each frame to be further processed to those data points shown in FIG. 12. As can be seen, in this example, the number of peaks in each frame relevant to the subject is just 10.

The 10 selected peaks were analysed across the frames of the data sample using FFT analysis. As discussed above, the accuracy of the FFT analysis to determine a heart rate and a respiratory rate is increased as the number of data points is increased, that is the number of frames in the data sample being analysed. FIG. 13 shows the results of the FFT analysis using 512 data points from 512 frames (n=512, that is 2x where x is 9). FIG. 14 shows the results of the FFT analysis using 1024 data points from 1024 frames (n=1024, that is 2* where x is 10). The FFT analysis was also conducted with values of n=256 and 2048.

After making a selection of the peaks in the FFT data related to heart rate and respiratory rate and carrying out the certainty estimation, the following results were obtained:

| No. of Data | Respiration | Certainty of | Heart | Certainty |
|---|---|---|---|---|
| Points (n) used | Rate | Respiration | Rate | of Heart |
| in FFT analysis | (Hz) | Rate | (Hz) | Rate |
| 256 | Not Detected | — | 1 | 0.7 |
| 512 | 0.35 | 0.6 | 1 | 0.8 |
| 1024 | 0.35 | 0.9 | 1 | 0.3 |
| 2048 | 0.35 | 0.4 | Not Detected | — |

The results indicate that respiration rate was not detected when the window size n was 256. This indicates that the window size n needed to be increased, as the frequency resolution obtained using such a low number was not sufficient to identify the relatively low frequency of the movements arising from respiration of the subject.

In contrast, the results indicate that the heart rate was not detected at the high values of window size n. This indicates that the frequency resolution was too high to identify the relatively high frequency movements arising from the heart of the subject beating.

Spectral leakage can occur at certain window sizes n, that is the energy of a frequency component spreading into an adjacent frequency. Spectral leakage can give rise to difficulties in identifying the respiration rate or heart rate of the subject. In such cases, alternative values for window size n should be selected.

Finally, the signal noise present at certain values of window size n may prevent an accurate indication of respiration and/or heart rate being obtained. Again, selecting a different window size n can reduce the noise in the signal, in turning allowing the respiration and heart rates to be detected.

The reduction in the certainty values at certain window sizes n may arise due to lower time resolutions leading to difficulty detecting transient events, such as heart beats or respiration; higher window sizes n requiring more computation, in turn increasing uncertainty or noise in the analysis; or larger window sizes n may cause the FT analysis to overfit the data, in turn leading to spurious peaks or noise being considered as significant peaks, reducing the certainty of the values. Again, altering the window size n can be used to optimise the certainty values.

As discussed above, the system and method of the present invention are also advantageously applied in detecting the movement of a subject. This may be used to detect a subject, for example in conditions where the visibility of the subject is obscured, such as by a structure or other material. The detection of movement of a subject can be achieved in a manner analogous to the method described above for monitoring the heart rate and respiration rate of a subject.

FIG. 15 shows a diagrammatical representation of one embodiment of the signal processing algorithm for processing signal data for determining the movement of a subject.

The input to the process are the signal data 602 received from the receiver of the system corresponding to detected reflected pulses of each frame. The sample period or window for detecting movement of the subject may be 5, 10 or 15 seconds. In the case of Embodiment A of the system and method, the signal data 602 are transmitted to the processor via a USB2 port. The signal data has a frame rate of 100 MHz, each frame comprising 512 samples of the reflected pulses, each sample having a 16 bit signal. Therefore, input signal data are transmitted at a rate of less than 1 Mbit/second.

The signal data 602 are passed to 6 first-in-first-out (FIFO) buffers 604, each comprising 512 registers. The registers have data sizes from 5,000 to 80,000 frames.

From the buffers 604, the signal data are subjected to a selection process, in which the peak signals relating to the subject being monitored of each frame are identified and selected. The selection process is as follows:

In a first selection stage 606, the signal data are analysed to determine the FIFO maximum, minimum and range of the signal data in each frame. To characterise the peaks in the detected pulses, the signal data are also subjected to full-width, half-maximum (FWHM) determinations.

There follows a subject selection stage 608. In operation, the system will detect a range of reflected pulses, some of which emanate from the subject of interest or being monitored, while others are reflected from other objects in the vicinity of the subject. To select the signal data relating to the subject, the distance of the object corresponding to the reflected pulses is determined. In addition the maximum signal strength of neighbouring peaks is determined. As discussed above, a human subject has a characteristic pattern of reflected pulses. In particular, a human subject may be identified by a strong first peak reflected pulse, corresponding to the front of the subject, that is the part of the subject closest to the transceiver, and a second, weaker peak reflected pulse emanating from some distance beyond the source of the first reflected peak. The second peak is phase correlated to the first, stronger peak. This distance is typically from 15 to 100 cm, depending upon the orientation of the subject relative to the transceiver. A typical pattern of peaks for a human subject is shown in FIGS. 6 and 7, as discussed above. By determining the signal thresholds, detecting the peak signals, making FWHM determinations of the peaks, as well as determining the distance corresponding to the time between the peaks and the relative signal range provides a characterisation of the object from which the reflected pulses emanate. This characterisation may be compared with a stored characterisation of a human subject, allowing those peaks in the signal data corresponding to a human subject to be identified and selected.

The quality of the signal is determined in the next selection step 610. The signal quality is determined by measuring the area under the curves of the signal peaks corresponding to a human subject and the total area under the curve for the entire set of data for each frame.

Finally, signal selection 612 is performed, selecting only those peak signals from each frame that correspond to a human subject. The selected signal data are then subjected to analysis to determine the heart rate and/or respiration rate of the subject. This is achieved using Fast Fourier Transform (FFT) analysis.

FFT analysis is performed in the FT Bank 614. FFT analysis is applied to the selected signal data on the basis of the following criteria, where ‘n’ is the number of frames in the data sample being analysed:

| 5-second | 10-second | 20-second | 40-second | 80-second |
|---|---|---|---|---|
| window | window | window | window | window |
| n = 256 | n = 256 | n = 256 | n = 256 | n = 256 |
| n = 512 | n = 512 | n = 512 | n = 512 | n = 512 |
| | n = 1024 | n = 1024 | n = 1024 | n = 1024 |
| | | n = 2048 | n = 2048 | n = 2048 |
| | | | n = 4096 | n = 4096 |

All signal data processed in the FT Bank 614 are subjected to FT Bank Averaging 616, in which the signal data are averaged. The averaged signal data are used to estimate the certainty of the final determination and in peak selection. Averaging of the signal data is as described hereinbefore.

Thereafter, movement of the subject is determined 618. Movement of the subject is a function of time and intensity. Movement of the subject is identified by comparing the peaks of one frame of the selected data set with the corresponding peaks from the previous and successive frames. Changes in the peaks over time indicates movement of the subject. For example, the peaks may change in strength (amplitude) and/or move in time.

The rate of movement 618 may also be determined by calculating the rate that changes in the peaks occur over the time of the pulse sampling.

A certainty estimation 620 is also performed by calculating the log FWHM of the maximum peaks corresponding to movement of the subject, divided by the total energy spectra of the sample set.

The output 622 of the process is an indication of the movement pattern of the subject, for example a rate of movement over time. These indications may be displayed to the user, for example by way of a display as shown in FIG. 1. Alternatively or in addition, these indications may be stored within the system and/or transmitted to a remote device.

The system and method of the present invention when applied to detecting the movement of a subject are further explained by way of the following working example, which is provided for illustrative purposes only.

### Example 2: Movement Detection

The system 402 was configured to operate according to Embodiment A described above.

The system 402 was configured to generate repeated trains of pulses, each train comprising 512 UWB radio-wave pulses (N=512) having a frequency between 6 and 8 GHz. A pulse was transmitted every 20 nanoseconds. Following the transmission of the first pulse P₁, the receiver was activated after a delay T₁ of 2 nanoseconds and operated to detect whether a reflected pulse is received. The receiver was activated after each successive pulse, T₂, T₃, T₄ . . . Tₘ₋₁, Tₘ . . . T₅₁₂, the delay in activating the receiver after the pulse transmission being increased incrementally by 26.5 picoseconds (that is ΔT₍ₘ₋₁₎₋ₘ is 26.5 picoseconds for all 512 pulses) up to a time T₅₁₂ of 15.6 nanoseconds.

This cycle of transmitting and receiving pulses was repeated up to 900 times per second, generating up to 900 frames per second of signal data.

Operating in this manner according to Embodiment A, the system was used to detect the movement of a subject behind cover. In this case, the subject was within a closed room and the system located outside the room and arranged to scan the room through a fire door rated at 60 minutes. The transmitter and receiver were positioned at a distance of 0.1 m from the fire door and oriented to direct pulses into the room normal to the door and receive reflected pulses.

The test was repeated three times: first with the subject standing still in the room; second with the subject walking in the room; and third with the subject running in the room. In each test, the subject was positioned at different locations in the room.

The signal data obtained from scanning the room with the subject present were analysed using the procedure shown in FIG. 15 and described above to detect movement of the subject.

The results are shown in FIG. 16, the key for which is as follows:

| Symbol | Description |
|---|---|
| ◯ | Positions in the room where the subject was stationary and |
| | was detected as a result of their breathing pattern; walking |
| | subjects were detected; and running subjects were detected. |
| □ | Positions in the room where the subject was stationary and |
| | was not detected; walking subjects were detected; and running |
| | subjects were detected. |
| ▪ | Positions in the room where the subject was stationary and was |
| | not detected; walking subjects were not detected; and running |
| | subjects were detected. |
| ● | Positions in the room where no movement was detected. |

The tests were repeated with the system oriented at different angles to the fire door. The results were similar to those shown in FIG. 16. The tests were also repeated with the system position adjacent the exterior surfaces of walls of the room. Walls of different construction were selected, including stud-partition walls, walls of breeze blocks, walls of breeze blocks and bricks, and walls of brick. In all cases, the system was able to detect the presence of a subject at different locations within the room, as a result of the movement of the subject.

In general, the subject was detected after a scanning period of from 10 to 15 seconds.

These tests demonstrate the ability of the system and method of the present invention to detect a subject when visibility of the subject is obscured and through a structure, such as a wall or a door.

Further tests were conducted under the conditions set out above, but with a plurality of subjects within the room. In these tests, the system and method were able to detect the presence of each subject in the room.

1. A system for scanning a subject comprising:
a transmitter operable to emit a train of pulses comprising a plurality of ultra-wide band (UWB) radio-wave pulses; and
a receiver operable to receive reflected radio-wave pulses corresponding to each pulse of the plurality of pulses emitted by the transmitter, the receiver operable to:
detect a reflected pulse for the first pulse of the plurality of pulses a time period T₁ seconds after the first pulse P₁ has been emitted; and
thereafter to detect a reflected pulse for each subsequent emitted pulse Pₘ, where m is the number of the pulse, a time period Tₘ seconds after the pulse Pₘ has been emitted, where Tₘ is longer than the time period Tₘ₋₁ for the preceding pulse Pₘ₋₁;
and
a control unit comprising a processor operable to receive data from the receiver relating to the reflected pulses detected.

2. The system according to claim 1, wherein the transmitter is operable to transmit a train of pulses, each pulse of which has a duration of 2 picoseconds or less.

3. The system according to claim 1, wherein the transmitter is operable to transmit pulses wherein the frequency of the radio waves of each pulse is in the range of from 7 to 7.5 GHz.

4. The system according to claim 1, wherein the transmitter is operable to transmit pulses at a rate of one pulse every 15 to 25 nanoseconds.

5. The system according to claim 1, wherein the increment ΔT₍ₘ₋₁₎₋ₘ is from 20 to 30 picoseconds.

6. The system according to claim 1, wherein the number of pulses in each train of pulses is from 450 to 550.

7. The system according to claim 1, wherein the transmitter and/or the receiver comprise a Vivaldi antenna.

8. A method of scanning a subject, the method comprising:
transmitting a train of pulses comprising a plurality of ultra-wide band (UWB) radio-wave pulses at the subject or an expected location of the subject;
detecting a reflected pulse for the first pulse of the plurality of pulses a time period T₁ seconds after the first pulse P₁ has been emitted;
detecting a reflected pulse for each subsequent emitted pulse Pₘ, where m is the number of the pulse, a time period Tₘ seconds after the pulse Pₘ has been emitted, wherein Tₘ is longer than the time period Tₘ₋₁ for the preceding pulse Pₘ₋₁; and
analysing the reflected pulses detected.

9. The method according to claim 8, wherein each pulse has a duration of 2 picoseconds or less.

10. The method according to claim 8, wherein the frequency of the radio waves of each pulse is in the range of from 7 to 7.5 GHz.

11. The method according to claim 8, wherein the pulses are transmitted at a rate of one pulse every 15 to 25 nanoseconds.

12. The method according to claim 8, wherein the increment ΔT₍ₘ₋₁₎₋ₘ is from 20 to 30 picoseconds.

13. The method according to claim 8, wherein the number of pulses in each train of pulses is from 450 to 550.

14. The method according to claim 8, wherein the receiver provides the processor with signal data constantly, the processor selecting a batch of signal data for the duration of the detection period corresponding to the period beginning time Tₘ after the pulse Pₘ has been transmitted.

15. The method according to claim 8, wherein signal data corresponding to the reflected pulses is analysed using Fast Fourier Transforms (FFT).

16. The method according to claim 8, wherein a plurality of trains of pulses is transmitted.

17. The method according to claim 8, wherein signal data corresponding to the reflected pulses is first analysed to select the portion of the signal data corresponding to the subject, thereafter further analysis being conducted only on the selected portion of the signal data.

18. The method according to claim 8, wherein signal data corresponding to the reflected pulses are analysed to determine the respiration rate and/or the heart rate of the subject.

19. The method according to claim 8, wherein signal data corresponding to the reflected pulses are analysed to determine the movement and/or location of the subject.
